(19) United States
(12) Patent Application Publication
(10) Pub. No.: US 2017/0175081 A1
(43) Pub. Date: Jun. 22, 2017

(54) Methods of Obtaining Islet Cells

(71) Applicants: The University Court of the University of Aberdeen, Aberdeen Aberdeenshire (GB); Lothian Health Board, Edinburgh (GB)

(72) Inventors: Kevin Docherty, Foresterhill Aberdeen (GB); Hilary Margaret Docherty, Foresterhill Aberdeen (GB); Maria Joao Marques De Lima, Foresterhill Aberdeen (GB); Kenneth Ross Muir, Foresterhill Aberdeen (GB); John Joseph Casey, Edinburgh (GB)

(21) Appl. No.: 15/310,121

(22) PCT Filed: May 14, 2015
(86) PCT No.: PCT/GB2015/051430

§ 371 Date: Nov. 10, 2016

(30) Foreign Application Priority Data
May 14, 2014 (GB) 1408570.8
Jul. 28, 2014 (GB) 1413329.2

(51) Int. Cl. C12N 5/071; A61K 35/39

(52) U.S. Cl. CPC C12N 5/0676; A61K 35/39; C12N 2501/60; C12N 2501/604; C12N 2506/1392; C12N 2510/00

(57) ABSTRACT

The present invention provides methods and materials relating to obtaining or expanding populations of islet cells, and uses of the islet cells obtained by these methods, for example in the treatment of diabetes. The invention uses transcription factors in a process of expansion and de-differention, followed by redifferentiation.

### TECHNICAL FIELD

The present invention provides methods and materials relating to obtaining or expanding populations of islet cells, and uses of the islet cells obtained by these methods, for example in the treatment of diabetes.

### BACKGROUND TO THE INVENTION

Diabetes is now recognized as a global epidemic that affects around 6% of the world's adult population. The International Diabetes Foundation Global Atlas predicts that the numbers will increase from 366 million in 2011 to 552 million in 2030.

There are two main forms of the disease; type 1 diabetes (T1D) and type 2 diabetes (T2D). Both are associated with decreased numbers of insulin secreting β-cells in the islets of Langerhans.

T1D is an autoimmune disorder in which activated CD4+ and CD8+ T lymphocytes infiltrate the islets and selectively destroy the β-cells. Although its onset is usually during infancy and puberty, it can occur at any age. The destruction of β-cells is initiated three or four years before the symptoms develop such that at the time of presentation up to 70-80% of the 8-cell mass is lost through apoptosis. T1D accounts for 5-10% of diabetes cases.

T2D results from a combination of insulin resistance and β-cell failure and is normally associated with being overweight or obese. It is particularly difficult to treat since the impaired actions of insulin lead to elevated blood levels of glucose and fatty acids, which in turn affect the function of the β-cell and in time, through inflammatory mechanisms, increase β-cell apoptosis. Very much a disease of middle-aged or elderly people, there has been an inexorable decrease in the age of onset of T2D associated with an increase in childhood obesity.

In the case of T1D, it is hoped that a cure may come from immune interventions directed at preventing the disease prior to the establishment of autoimmunity (Thrower and Bingley, 2011). Although several immunotherapeutic targets have been identified, there are still major challenges in setting up and evaluating vaccine trials (Skyler, 2013).

In the meantime improved insulin therapy, with emphasis on closed loop delivery systems or islet transplantation, is generally accepted as the best way forward. A comparison of continuous glucose monitoring data from patients on closed loop delivery systems and those that have undergone islet transplants indicates that close loop delivery systems cannot get close to matching the control that can be achieved by islet transplantation.

Islet transplantation, mainly in the context of syngeneic transplantation following removal of the pancreas in patients with pancreatitis has been around since the early 1990's (McCall and Shapiro, 2012). The success rate for syngeneic islet transplants has been relatively good, but allogeneic transplantation of donor islets for the treatment of T1D was plagued from the outset with poor success rates; 8% graft function after one year.

This changed with the introduction of the Edmonton Protocol in 2000, which placed emphasis on transplanting a sufficiently large number of islets, minimizing the cold ischemia time and changing the immunosuppressive regimen and in particular avoiding the use of steroids that are known to affect islet cell function (Shapiro et al., 2000). The introduction of the Edmonton protocol in 2000 demonstrated that human donor islet transplantation can lead to a significant decrease in exogenous insulin requirements and even temporary insulin independence along with reduction of severe hypoglycaemia (Shapiro et al., 2000)

With further improvements in immunosuppression, clinical islet transplantation has progressed considerably such that by the end of 2013 over 750 patients with T1D have received transplants. The one-year success rates are high, although there are still concerns about graft failure with time (McCall and Shapiro, 2012). Priority is given to patients who are C-peptide negative, and who have displayed severe episodes of hypoglycemia and reduced ability to detect the symptoms of impending hypoglycemia.

As previously mentioned, the success of the Edmonton Protocol is in part due to the transplantation of a large islet mass (>11,000 IEG/Kg), which can often be best achieved using islets from multiple donors (average 2-3). The lack of donor material is a significant problem as the protocol relies on the availability of large quantities of donor islets.

It can thus be seen that novel methods of providing islet materials, including (but not limited to) insulin secreting β-cells, would provide a contribution to the art.

### DISCLOSURE OF THE INVENTION

The present invention provides, inter alia, methods, uses and kits for obtaining expanded populations of islet cells. The invention thus has utility, inter alia, for providing increased quantities of islet material for use in transplantation.

It was previously known that when human islets are placed in long term adherent culture conditions, fibroblast-like cells migrate out from the islet foci (Gershengorn et al., 2004). These cells can proliferate and form a monolayer that can be grown to passage 20 and beyond. A similar scenario occurs when the islets are dispersed and plated as single cells (Russ et al., 2008). Formation of the fibroblast-like monolayer is accompanied by loss of epithelial markers, including insulin and other endocrine hormones, and acquisition of mesenchymal markers, suggesting that the islets dedifferentiate via a process that bears the characteristics of epithelial to mesenchymal transitioning (EMT) (Gershengorn et al., 2004). Moreover, the fibroblast-like cells express cell surface markers (CD90, CD105 and CD73) of mesenchymal stromal cells (MSCs) and can be induced to redifferentiate towards osteoclasts, chondrocytes and adipocytes.

The present inventors have previously shown that human exocrine-enriched cells can be efficiently reprogrammed into functional β-like cells, using a combination of four pancreatic transcription factors, namely PDX1, MAFA, NGN3 and PAX4 (Lima et al., 2013). The protocol for producing functional β-like cells only worked when EMT was suppressed using a combination of TGFβ1 and Rho-kinase inhibitors. Other transcription factors may also play a role in pancreatic cell reprogramming (Zhang et al., 2012; Jonghyeob et al., 2013).

As described in more detail in the Examples below, the present inventors have shown that, unexpectedly, Krüppel-like factor 4 (KLF4) can induce a mesenchymal-to-epithelial transition (MET) i.e. a reversal of the EMT dedifferentiation process described above.

The MET is evidenced by upregulation of epithelial markers and down-regulation of mesenchymal markers.

The ability to induce MET in dedifferentiated pancreatic tissue allowed the present inventors to go on to show that MSCs derived from β-cells and those from acinar cells were functionally equivalent in terms of their ability to dedifferentiate towards endocrine and exocrine lineages.

The inventors have shown that the beta-like cells produced by the MET re-express insulin. Other endocrine and exocrine markers present in the original differentiated islet enriched cell population are also recovered. These findings hold promise that cells, for example, beta cells which have dedifferentiated and expanded ex-vivo can be redifferentiated toward beta cells.

These finding have important implications for cell therapy approaches to the treatment of type-1 diabetes since dedifferentiation, expansion, and redifferentiation of islet or pancreatic tissue left over from the islet isolation procedure could provide a potentially unlimited supply of islets for transplantation (Muir et al., 2014). The results have potential to address a much needed requirement for a replenishable population of beta cells suitable for human transplantation.

Thus in one aspect the invention relates to the use of KLF4 to induce differentiation (or more particularly re-differentiation) of pancreas-derived MSCs, for example in the methods described herein.

Yori et al. (2010) previously described the effects of KLF4 on E-cadherin gene expression and conclude that KLF4 has a role in preventing EMT in mammalian epithelial cells, suggesting a metastasis suppressive role for KLF4 in breast cancer. However that publication did not teach or suggest that KLF4 could be used to re-differentiate of pancreas-derived MSCs.

Thus disclosed herein are methods for the expansion of pancreatic cells, for example islet cells.

More specifically methods of the invention may comprise ex-vivo expansion of pancreatic cells. The methods involve a step of dedifferentiation/EMT and expansion, followed by a step of MET/redifferentiation.

In preferred embodiments the methods allow expansion of dedifferentiated pancreatic cells several thousand-fold in monolayer and then induction of redifferentiation.

In one aspect the invention provides a method of expanding a population of pancreatic cells, the method comprising:
a) culturing the pancreatic cell population in conditions that promote expansion and dedifferention; then
b) inducing redifferentiation
c) obtaining redifferentiated pancreatic cells.

The invention also provides a method of obtaining pancreatic cells, the method comprising:
a) providing a pancreatic islet cell population
b) culturing the pancreatic cell population in conditions that promote expansion and dedifferention; then
c) inducing redifferentiation.
d) thereby obtaining redifferentiated pancreatic cells.

The invention also provides a method of producing an expanded population of pancreatic cells, the method comprising:

(i) providing a starting pancreatic cell population;

(ii) culturing the starting pancreatic cell population under a first condition that promotes expansion and dedifferention of the starting islet population;

(iii) culturing the cells obtained in step (ii) under a second condition which induces redifferentiation;

(iv) thereby obtaining an expanded population of redifferentiated pancreatic cells.

The invention also provides a method of obtaining pancreatic cells, or populations of pancreatic cells, the method comprising induction of differentiation of pancreas-derived mesenchymal stromal cells (MSCs).

The invention also provides methods of inducing redifferentiation of pancreatic cells following expansion in culture.

Redifferentiation in the above methods and uses (e.g. the ‘second condition’ described above) can be induced by culturing the cells in the presence of KLF4 (e.g. exogenous KLF4).

Thus the invention also provides a method of inducing MET in dedifferentiated pancreatic cells, the method comprising introducing into the cells a nucleic acid or protein preparation which expresses or provides a transcription\differentiation factor which is KLF4.

Starting Materials

As is described in more detail in the Examples, the present inventors have shown for the first time that MSCs derived from insulin positive β-cells or amylase-positive acinar cells are functionally equivalent, in that they can both be induced by Ad-KLF4 to express endocrine and exocrine markers and have the feature common to all MSCs populations of being able to differentiate towards adipose and osteogenic lineages.

The Examples demonstrate that left over islets or exocrine tissues can both have utility for providing expanded cell populations useful in therapy.

The cells for use in the methods of the present invention may comprise exocrine and/or endocrine pancreatic cells. The cells for use in the methods of the present invention may comprise passenger stromal cells.

Although the cells may be any mammalian cells (e.g. primate, rodent, porcine, bovine, canine, equine, feline, and so on)preferably, the cells for use in methods the present invention are human pancreatic cells, for example epithelial cells. In preferred embodiment the cells for use in the methods of the present invention comprise islet cells. Islet cells for use in the present invention can be obtained, for example from human donor pancreases.

A preferred starting material is an Islet enriched fraction (IEF) of the pancreas. An islet cell population which can be used in the methods of the invention will generally include beta cells (β-cells) plus optionally acinar cells such as amylase positive acinar cells, alpha cells (α-cells) and other epithelial cell types of the pancreas.

The starting cell population is cultured ex-vivo in conditions described herein to carry out the methods of the invention.

Once the cells have been dedifferentiated, they can be passaged ‘indefinitely’ so that fresh cadaveric tissue is not required, thus providing a replenishable supply of pancreatic cells for use in the present invention.

Cells Obtainable by the Methods

A preferred product of the method is, or comprises, redifferentiated islet cells which are beta-like cells which express insulin mRNA. The product may comprise other epithelial cells of the pancreas. As noted above, the inventors have shown that descendants of both beta cells and acinar cells can be redifferentiated by KLF4 into beta-like cells. This suggests descendants of all epithelial cell types (including potentially passenger stromal cells) can be redifferentiated to beta-like cells.

Although the redifferentiated beta-like cells characterised by the inventors expressed lower levels of insulin mRNA than native beta cells, it will nevertheless be appreciated that these cells still have utility, for example in therapeutic interventions in diabetes. In particular a replenishable source of insulin-producing cells is an important advancement in the treatment of diabetes.

In other embodiments a preferred product of the method is, or comprises, redifferentiated islet cells which are delta-like cells that express somatostatin mRNA. Delta-like cells may produce somatostatin protein. Other pancreatic epithelial cells may also be obtained by methods of the present invention.

In other embodiments a preferred product of the method is, or comprises, redifferentiated islet cells which are alpha-like cells that express glucagon mRNA. Alpha-like cells may produce glucagon protein.

In other aspects, the invention provides pancreatic cells or pancreatic cell populations obtained or obtainable by the methods described herein. It further provides use of these in methods of treatment, for example methods of treating diabetes.

These and other embodiments and aspects of the present invention will now be discussed in more detail. Any sub-titles herein are included for convenience only, and are not to be construed as limiting the disclosure in any way.

Dedifferentiation

The dedifferentiation step in the methods described herein may also be referred to as epithelial-mesenchymal transition (EMT).

As explained above, EMT is a process whereby epithelial cells that are normally non-proliferative and non-mobile undergo transition into mesenchymal cells (sometimes referred to herein as mesenchymal stromal cells (MSCs) characterized by a proliferative and mobile phenotype. EMT is, therefore, a process of disaggregating epithelial units and re-shaping epithelia for movement in the formation of mesenchymal cells.

The mesenchymal stromal cells (MSCs) that result from EMT, have high proliferative capacity but are devoid of any hormone production. The markers used to identify such cells and EMT are described in more detail elsewhere herein. Culture conditions suitable for promoting dedifferentiation and expansion are also described in more detail elsewhere herein.

Redifferentiation

Redifferentiation may be referred to herein, unless context demands otherwise, as differentiation or mesenchymal-epithelial transition (MET).

It has previously been shown that when human islet-derived MSCs are transferred from serum-containing to serum-free medium, the cells form epithelial like clusters and re-express low levels of endocrine hormones (Davani et al., 2007; Gershengorn et al., 2004). This effect can be enhanced by addition of soluble factors or by targeting components of the EMT signaling pathway (Bar et al., 2008; Bar et al., 2012; Ouziel-Yahalom et al., 2006).

The present inventors have shown for the first time that KLF4 overexpression initiates MET and redifferentiation of human pancreatic cell types. MET represents the reversal of the dedifferentiation process.

Genetic lineage tracing studies demonstrated that mesenchymal cells derived from β-cells or amylase-positive acinar cells could be redifferentiated by KLF4 into both endocrine and exocrine lineages.

Accordingly, the methods of the present invention involve a step of redifferentiation, by inducing MET. This step involves redifferentiation toward epithelial cells, more specifically pancreatic cell types as described above.

In preferred embodiments the methods involve induction of redifferentiation toward beta cells.

Utilities for KLF4

In the methods, induction of redifferentiation may involve introducing a nucleic acid or protein preparation which expresses or provides one or more transcription factors into the cells. In particular, induction of redifferentiation may involve introducing a nucleic acid or protein preparation which expresses or provides KLF4 into the cells.

In the methods, induction of redifferentiation may involve culturing the cells in the presence of one or more transcription factors. In particular, induction of redifferentiation may involve culturing the cells in the presence of KLF4. Where the methods involve culturing the cells with protein preparations, the culturing allows the transcription factors to be taken up by the cell.

In some embodiments of the method, induction of redifferentiation involves contacting the cells with a protein preparation of KLF4.

In alternative embodiments induction of redifferentiation involves expressing the KLF4 in the cells.

The expressions ‘culturing the cells in the presence of . . . ’, ‘culturing the cells in media comprising . . . ’, ‘treating cells with . . . ’, ‘contacting the cells with’ and ‘introducing . . . into the cell’ are used interchangeably, unless context demands otherwise.

The expressions ‘expressing . . . in the cell’ are used interchangeably with ‘introducing a nucleic acid which expresses . . . ’ in method steps of the present invention, unless context demands otherwise.

Examples of suitable expression vectors for expressing KLF4-encoding nucleic acid in a cell are discussed in more detail hereinafter.

The cells may be cultured with the one or more transcription factors (e.g. KLF4) for 2 to 10 days or longer, for example, 2, 3, 4, 5, 6, 7, 8, 9 or 10 days. In preferred embodiments, the cells are cultured with the transcription factor(s) for 4 or more days.

The transcription factor KLF4, one of 17 Krüppel-like factors, is a member of a family of proteins characterized by their three Cyst His2 zinc fingers located at the C-terminus, each of which is separated by a highly conserved H/C link.

The sequence of the human KLF4 nucleic acids and protein are available, for example, in GenBank under NM_004235.4, NP_004226.3 and UniProt accession number O43474 (version 132).

KLF4 has been shown to have roles in processes from terminal differentiation in development, maintenance of a pluripotent state and progression of cancers. In particular KLF4 has been shown to play a role in reprogramming human somatic cells into iPSCs.

It is believed that MET may be an early and essential process in the generation of iPSCs from murine fibroblasts using the transcription factor cocktail Oct-4, Sox2, KLF4 and c-Myc (Takahashi and Yamanaka, 2006; Li et al., 2010; Samavarchi-Tehrani et al., 2010). KLF4 may be important in the MET process. When KLF4 is overexpressed in the absence of the other transcription factors, epithelial markers were up-regulated significantly (Samavarchi-Tehrani et al., 2010). Furthermore, KLF4 was shown to be bound by the E-cadherin promoter (Koopmansch et al., 2013; Yori et al., 2010) and to act as a critical regulator of genes critical for EMT, including SLUG (Lin et al., 2012; Liu et al., 2012) and JNK1 (Tiwari et al., 2013). KLF4 is significantly down-regulated in cells undergoing EMT (Lehembre et al., 2008; Tiwari et al., 2013).

However, in the context of the present invention, the lineage-specific differentiation demonstrated by the present inventors, provides a replenishable supply of beta cells or other pancreatic cells through targeting pathways required for MET but bypassing pluripotency and its associated risks.

Other Transcription Factors Having Utilities in the Method

The methods of the invention are practised by expressing or providing a transcription factor to a culturing islet cell population. More than one transcription factor may be expressed or provided, as redifferentiation agent. This agent may consist, or consist essentially of KLF4, or may comprise more than one transcription factor.

For example KLF4 may be used in combination with other transcription factors in inducing (re)differentiation in the methods of the present invention. The factors may affect the initiation, maturation and/or stabilisation of MET in mesenchymal stromal cells (MSCs) derived from pancreatic tissue.

The factors which may be used in combination KLF4 may be selected from the list consisting of: FOXA1, FOXA2, PDX1, NGN3, PAX4, MAFA, NKX6.1, NKX2.2, NEUROD1, PAX6, IA-1 and GATA4.

“Combination” in the present context embraces both the use of the factor or factors simultaneously or sequentially with KLF4.

Thus the agent may consist of, or consist essentially of, 1, 2, 3, 4, 5, 6, 7, 8, 9, 10, 11 or 12 of these factors in combination with KLF4.

One preferred agent is: KLF4+FOXA1

Another preferred agent is: KLF4+FOXA2

In each case it is preferred that the combination is used sequentially.

In some embodiments the transcription factors used in combination with KLF4 comprise, consist or consist essentially of PDX1, MAFA, NGN3 and PAX4. In some embodiments of the methods the transcription factors comprise PDX1, MAFA, NGN3 and PAX4, but do not comprise NKX6.1 and/or ND1.

Example Culture Conditions

Isolated human islets of Langerhans can be maintained as functional units in suspension culture for long periods of time without proliferation (Andersson et al., 1976; Nielsen et al.,1979).

As discussed hereinbefore, when human islets are placed in adherent culture conditions, MSCs are generated. Culture-expanded MSCs consist of a heterogeneous population of cells exhibiting a spectrum of phenotypes and functional properties (Zanini et al., 2011), and the extent of this heterogeneity is dependent on the tissue, donor and species of origin, isolation technique, culturing protocols, media used, and passage number (Ankrum et al., 2014; Jaager et al., 2014).

There is some controversy concerning the origins of the MSCs that occur when islets are placed in culture. Genetic lineage tracing studies in mice showed that β-cells dedifferentiated in culture but failed to proliferate and were eliminated from the culture (Chase et al., 2007; Morton et al., 2007; Weinberg et al., 2007). However, genetically traced cultured human β-cells dedifferentiate and replicate (Lima et al., 2013; Russ et al., 2008; Russ et al., 2009). It is believed that the MSC population arises from dedifferentiated epithelial cells via a process of EMT as well as from passenger stromal cells.

Irrespective of this, embodiments of the present invention conditions which promote expansion and differentiation will preferably involve culturing the cell population in adherent culture conditions.

In some embodiments the cells are cultured on laminin, such as laminin isoforms LN111, LN211, LN332, LN411, LN421, LN511 and LN521. The laminin isoform may be LN511 or LN521, e.g. LN521.

In the expansion step, the cells may be cultured in serum containing medium. For example, the cells may be cultured in RPMI with 10% FBS.

The cells may be grown in conditions which promote expansion and differentiation long term, for example for 1, 2, 3, 4, 5 or more weeks. The cells may be grown in conditions which promote expansion and differentiation long term, for example for up to 5, 10, 15, 20, 25, 30, 35 of 40 days. The cells may be passaged every 5 to 7 days.

The cells may be cultured in these conditions until they have expanded at least 10, 100, 1000, or 10′000-fold.

Once the cells have been dedifferentiated, they can be passaged ‘indefinitely’ so that fresh cadaveric tissue is not required.

In some embodiments redifferentiation may be induced at low passage number, i.e. the cells may be passaged only a few times before redifferentiation, for example, 1-8 times. The cells may be passaged, for example, 1-6, 2-5, or 2-3 times before redifferentiation is induced. The cells may be passaged 1, 2, 3, 4, 5, 6, 7 or 8 times before redifferentiation is induced. The cells may be passaged less than 8, for example, less than 5 times prior to induction of redifferention.

The present inventors have shown that suspension culture enhances redifferentiation. More specifically, the Examples show that suspension culture after KLF4 transduction enhanced Ecad, Epcam and decreased vimentin and SLUG expression compared to cells transduced with a control ADGFP adenovirus (FIG. 5). Pdx1, NGN3, amylase and CK19 were all enhanced in suspension culture (FIG. 5). Other cell markers for monitoring differentiation status are discussed in more detail below.

The present inventors have also shown redifferentiation in suspension culture allows exocrine and endocrine gene expression to be maintained, i.e. the transient nature of redifferentiation is overcome (Example 2; FIG. 16).

Accordingly, in some preferred embodiments of the present invention induction of redifferentiation is carried out in suspension culture. Alternatively, redifferentiation may be carried out in adherent culture. The cells may be cultured on laminin, for example on laminin coated plates. The cells may be cultured on laminin throughout the method. For example, the cells may be cultured on laminin comprising, consisting or consisting essentially of laminin isoform LN521.

In the redifferentiation step, the cells may be cultured in serum free medium, for example with RPMI supplemented with 1% BSA and 10 ug/ml insulin, 5.5 ug/ml transferrin and 6.7 ng/ml sodium selenite.

The cells may be incubated in suspension/adherent culture for 2 to 10 days or longer, for example, 2, 3, 4, 5, 6, 7, 8, 9 or 10 days. In some preferred embodiment the cells are incubated in suspension culture for about 6-10 days, for example 8 days.

In preferred embodiments the cells are placed in suspension culture about 0-3 days, e.g 1, 2 or 3 days after treatment with the transcription factor(s). Preferably, the cells are placed in suspension culture about 1 day after treatment with the transcription factor(s) (e.g. including KLF4).

In other embodiments the cells are placed in adherent culture about 0-3 days, e.g 1, 2 or 3 days after treatment with the transcription factor(s). The cells may be placed in adherent culture about 1 day after treatment with the transcription factor(s) (e.g. including KLF4).

Another aspect of the present invention is the use of suspension culture conditions to enhance differentiation (MET). The differentiation is carried out using factors and conditions as described herein.

Markers for Cell Types

The induced EMT and MET in the context of the methods of the present invention can be assessed and monitored using markers and morphological changes. MET is as evidenced by upregulation of epithelial markers and down-regulation of mesenchymal markers, while EMT is evidenced by upregulation of mesenchymal markers and down-regulation of epithelial markers. Morphological changes between cell types may also be used.

Redifferentiation in the context of the present invention is also evidenced by upregulation of endocrine hormones, endocrine transcription factors, acinar markers and ductal markers, which indicate differentiation into pancreatic cells types.

Exemplary markers and changes are described below. One or more of these markers or changes may be used to monitor MET/differentiation.

Islet cells obtained by the methods of the present invention may express one or more markers associated with MET, including upregulated epithelial markers, down-regulated of mesenchymal markers, morphological changes, upregulated endocrine hormones, endocrine transcription factors, acinar markers and ductal markers.

In some embodiments, at least 40%, at least 45%, in particular at least 50% of the cells in the cell population obtained express markers associated with differentiation (e.g. epithelial markers) as opposed to mesenchymal markers. In some embodiments, at least 40%, at least 45%, in particular at least 50% of the cells in the cell population obtained express markers associated with differentiation. In some embodiments, at least 40%, 45%, 50%, 55% of cells obtained by the process express ECAD. In a preferred embodiment at least about 50% of cells obtained express ECAD.

In some embodiments, the cells obtained from the methods of the invention include beta-like cells capable of insulin production. Beta-like cells may express one or more of the markers or changes that are associated with MET (as well as insulin expression). In some embodiments the cells obtained include δ-like cells that express somatostatin. Other pancreatic epithelial cells may be obtained by methods of the present invention.

In all cases these markers can be monitored by monitoring gene expression, for example using real-time quantitative PCR. Alternatively, morphological changes and protein distribution of the markers can be assessed by immunocytochemistry, for example florescence immunocytochemistry.

Epithelial Markers Include E-Cadherin and EPCAM

Mesenchymal markers include vimentin, α-smooth muscle actin (α-SMA), Snai2 (SLUG) and Zeb-1.

Other changes that occur in MET include morphological changes. During MET cells undergo a transition to a more rounded epithelial form.

Other markers of differentiation into pancreatic cell types include the acinar marker amylase and ductal marker CK19.

Additionally endocrine hormones insulin (INS) (or C-peptide), somatostatin (SST) and glucagon (GCG) are markers of differentiation into pancreatic endocrine cells. Specifically, C-peptide or Insulin are markers of beta cells, SST is a marker of delta cells, and GCG is a marker of alpha cells.

Endocrine transcription factors NGN3, MAFA, NKX6.1, NeuroD1 and PDX1 (transcription factors present in developing and mature beta cells) may also act as markers of differentiation.

Treatment with Zinc

The present inventors have shown that treatment of the cells with zinc increases both the level of insulin mRNA expression and the C-peptide content of the cells during reprogramming.

Accordingly, in preferred embodiments the cells are treated with zinc. For example the cells may be treated with ZnCl₂. Zinc (e.g. ZnCl₂) may be added concurrently with the transcription factor KLF4.

Zinc (e.g. ZnCl₂) may be added at a concentration of about 0.1 μM to about 100 μM, for example from about 1 μM to about 20 μM, about 5 μM to about 15 μM, about 8 μM to about 12 μM. Preferably zinc (e.g. ZnCl₂) is added at a concentration of about 10 μM.

In another aspect the invention relates to the use of zinc to enhance differentiation (MET) of pancreatic cells, where the differentiation is carried out using factors and conditions as described herein.

Further Methods and Conditions

The effects of KLF4 in Example 1 were transient, suggesting that other factors or conditions may be usefully applied to complete the MET programme and maintain cells in an epithelial state. This is reminiscent of the events that occur during OSKM-mediated reprogramming towards iPSCs (Takahashi and Yamanaka, 2006), where three consecutive phases have been identified; initiation, maturation and stabilisation (Samavarchi-Tehrani et al., 2010). Most studies suggest that KLF4, and to some extent c-Myc, is the key driver of the initiation phase. Similarly, in 3T3L1 cells that have been induced to differentiate towards adipocytes, KLF4 is expressed within 30 min and peaks at around 2 h after induction. It appears to act upstream of the major differentiation factors C/EBPβ and PPARγ2 (Birsoy et al., 2008). This very early transient pattern of expression is compatible with a role for KLF4 in promoting MET in 3T3L1 cells.

The present inventors have shown that transiency can be overcome by using suspension culture (Example 2).

In addition to treatment with KLF4, the cells may be treated with other factors, or cultured in conditions as detailed herein, for example to stabilize the effect of KLF4 (i.e. to maintain the cells in an epithelial state). The cells may be treated with other factors, and/or cultured in conditions, for example to preferentially redifferentiate the cells toward beta-like cells.

For example, the cells may be treated with an inhibitor of ARX expression and/or function for these purposes. The cells may be treated with the transcription factors PDX1, MAFA, NGN3 and PAX4 for these purposes. The cells may be cultured at low glucose concentrations for these purposes. The cells may be cultured on laminin instead of in suspension culture. Preferred conditions for such treatments are detailed elsewhere herein.

Accordingly, in some embodiments of the invention the method comprises, consists or consists essentially of:
a) providing a pancreatic islet cell population
b) culturing the pancreatic cell population in conditions that promote expansion and dedifferention; then
c) inducing redifferentiation by:
   (i) treating the cells with one or more transcription factors including KLF4 and optionally PDX1, MAFA, NGN3 and PAX4; then optionally:
      i. culturing the cells with betacellulin, exendin-4 and/or nicotinamide, and
      ii. treating the cells with an inhibitor of ARX expression and/or function
d) thereby obtaining redifferentiated pancreatic cells.

Step (c) may be carried out at low glucose concentration. Steps (i) and (ii) may be carried out in suspension culture.

Preferred culture times and conditions, such as glucose concentration and redifferentiation culture, are detailed elsewhere herein. An exemplary method is detailed in FIG. 13.

In the methods of the present invention redifferentiating the cells may comprise inhibition of ARX expression and/or function. Preferably the cells are also treated with transcription factors comprising, consisting or consisting essentially of: PDX1, MAFA, NGN3 and PAX4.

In the methods zinc (e.g. ZnCl₂) may be added with the transcription factors (e.g. KLF4 and PAX4). For example, zinc may added with KLF4, PDX1, MAFA, NGN3 and PAX4.

Zinc (e.g. ZnCl₂) may be added concurrently with inhibition of ARX. In some embodiments, treatment with transcription factors, inhibition of ARX and treatment with zinc are all concurrent.

Details of the human ARX and its protein and nucleotide sequences can be found at Uniprot (Accession number: Q96QS3 (version 120)) and Genbank (accession number and version: NM_139058.2).

Inhibition of ARX expression and/or function may comprise inhibition of: transcription of the gene, RNA maturation, RNA translation, post-translational modification of the protein, binding of the protein to a target. Inhibition may be conducted by an inhibitor that is a nucleic acid, a polypeptide, a protein, a peptide or a chemical compound.

The term “expression” when used in the context of expression of a gene or nucleic acid refers to the conversion of the information, contained in a gene, into a gene product. A gene product can be the direct transcriptional product of a gene (e.g., mRNA) or a protein produced by translation of a mRNA. Gene products include messenger RNAs which are modified, by processes such as capping, polyadenylation, methylation, and editing, and proteins (e.g., ARX) modified by, for example, methylation, acetylation, phosphorylation, ubiquitination, SUMOylation, ADP-ribosylation, myristilation, and glycosylation.

Inhibition of ARX expression may be by using antisense nucleic acid capable of inhibiting transcription, or translation of the corresponding messenger RNA. The antisense nucleic acid can comprise all or part of the sequence of ARX, or of a sequence that is complementary thereto. The antisense sequence can be a DNA, and RNA (e.g. siRNA) or a ribozyme. In a preferred embodiment ARX expression is inhibited by small inhibitory RNA (siRNA). Nucleic acids including RNAs can be transduced into the cells using vectors, such as viral vectors. In some embodiments the cells are transduced with siRNA. Methods of inhibiting ARX expression are discussed in more detail hereinafter.

Inhibition of ARX may be carried out during treatment with the transcription factor(s) (e.g including KLF4. For example, inhibition of ARX may be carried out at 0-1, 1-2, 2-3, 3-4 or 4-5, 5-6, or 6-7 days after treatment with the transcription factor(s) begins. For example about 1, 2, 3, 4, 5, 6 or 7 days after treatment with the transcription factor(s) begins. Inhibition of ARX may be carried out about 6 or 7 days after treatment with the transcription factor(s) begins. Inhibition of ARX may be carried out about 6 or 7 days after the redifferentiation step begins.

The methods of the invention may involve culturing the cells in the presence of one or more of betacellulin, exendin-4 and nicotinamide. In some embodiments the method involves culturing the cells in the presence of all of betacellulin, exendin-4 and nicotinamide (BEN). In some embodiments treatment with one or more of betacellulin, exendin-4 and nicotinamide follows culture with the transcription factor(s). In some embodiments there is overlap between culture with one or more of betacellulin, exendin-4 and nicotinamide and the transcription factor(s). In some embodiments the cells are cultured simultaneously with transcription factors and one or more of betacellulin, exendin-4 and nicotinamide.

In some embodiments the cells are cultured in the presence of one or more of betacellulin, exendin-4 and nicotinamide for 3-10 days, for example, 5-9 days, preferably about 8 days.

Betacellulin, exendin-4 and/or nicotinamide may be added for example 0-3, e.g. about 1, 2 or 3 days (preferably 1 day) after treatment with the transcription factors begins. Preferably the cells are cultured in the presence of betacellulin, exendin-4 and/or nicotinamide for a time frame overlapping with treatment with the transcription factor(s). For example the cells may be cultured for 8 days with BEN overlapping with treatment with the transcription factor(s).

The cells may be suspended in culture supplemented with betacellulin, exendin-4 and/or nicotinamide after treatment with transcription factor(s), for example about 1 day after treatment with the transcription factor(s).

Recent studies in mice have shown that glucose metabolism is a key regulator of compensatory β-cell proliferation (Porat et al., 2011). Porat et al. propose a mechanism for homeostasis of beta-cell proliferation and mass involving adjustment of proliferation according to the rate of glycolysis.

The cells may be cultured in low glucose concentrations. The cells may be cultured in low glucose concentrations throughout the redifferentiation step. For example, the cells may be cultured in low glucose concentrations for about 5-15 days or 6-12 days, for example for about 8 days.

The glucose concentration level may be between 0-5 mM, for example between 0.5-4.5 mM, 1-5 mM, 1-4.5 mM, 1-4 mM, 1.5-4.5 mM, 1.5-4 mM. In particular the glucose concentration may be between 2-4.5 mM, 2-4 mM, 2-3 mM. In one embodiment the cells are cultured in a concentration of about 2.5 mM glucose.

In one aspect the invention provides use of low glucose culture (e.g. concentrations of 5 mM or less) to stabilize the effects of KLF4 of redifferentiating cells (e.g. toward beta-like cells). Use of the low glucose concentration culture may be in conjunction with the conditions, including factors and agents that are used in the methods of reprogramming described herein.

Treatments and Other Utilities

The islet cells (for example including beta-like cells) obtained by methods of the present invention may be used to produce insulin, preferably in vivo or ex vivo.

The islet cells (for example including alpha-like cells) obtained by methods of the present invention may be used to produce glucagon, preferably in vivo or ex vivo.

As mentioned above, the success of the Edmonton Protocol is in part due to the transplantation of a large islet mass (>11,000 IEG/Kg), which can often be best achieved using islets from multiple donors (average 2-3). The methods described herein may allow a large supply of culture-expanded allogeneic MSCs from a single donor to be redifferentiated and used to treat a large number of diabetic patients. The methods described herein may be used to generate clinically meaningful numbers of β-like cells.

Thus the islet cells (for example including beta-like cells) obtained by methods of the present invention have particular utility in clinical situations to treat diabetes.

The cell population obtained by the methods may be used directly, or optionally may be subject to further steps, for example to prepare the cells population for clinical use, or to enrich it for certain cells (e.g. cells capable of producing insulin). Furthermore sub-sets of epithelial cells may be isolated from the population for use as required.

Therefore, the present invention includes islet cells obtained by the methods described herein for use in a method of treatment by therapy, for example for treating diabetes in a patient.

The term “treatment,” as used herein in the context of treating a condition, pertains generally to treatment and therapy of a human, in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included. “Prophylaxis” in the context of the present specification should not be understood to circumscribe complete success i.e. complete protection or complete prevention. Rather prophylaxis in the present context refers to a measure which is administered in advance of detection of a symptomatic condition with the aim of preserving health by helping to delay, mitigate or avoid that particular condition.

Patients to be treated include those suffering from (diagnosed with) diabetes.

Treatment of diabetes in the context of the present invention may be treatment of type-1 diabetes or other causes leading to insulin deficiency e.g. post pancreatectomy. The treatment may also be of type-2 diabetes.

In some embodiments the patients to be treated may be C-peptide negative.

Additionally or alternatively, the patient may display, or have displayed, severe episodes of hypoglycaemia and/or reduced ability to detect the symptoms of impending hypoglycaemia.

The islet cells can be delivered in a therapeutically-effective amount.

The term “therapeutically-effective amount” as used herein, pertains to that amount of the receptor or ligand which is effective for producing some desired therapeutic effect, such as restoration of hypoglycaemic awareness, or independent of the need for external insulin, commensurate with a reasonable benefit/risk ratio, when administered in accordance with a desired treatment regimen.

Thus the invention also relates to methods of treatment of diabetes using islet cells obtained by the methods described herein.

The invention also relates to use of islet cells obtained by the methods described herein for use in the preparation of a medicament for treatment of diabetes.

Islet cells obtained by the methods described herein may be administered to a patient, for example they may be used in cell or cellular therapy. The islet cells obtained by the methods described herein may be transplanted into patients. Such cells may be manipulated before use e.g. encapsulated. The cells may be utilised in an external or implantable device or container.

Preferably the treatment is based on the Edmonton Protocol and may comprise the steps of infusing the islet into the patient, for example the patient's portal vein, optionally in conjunction with one or more (e.g. two) immunosuppressants (for example sirolimus and tacrolimus) and\or a monoclonal antibody intended to prevent organ rejection (for example daclizumab). The particular protocol would be at the discretion of the physician who would also select dosages using his/her common general knowledge and dosing regimens known to a skilled practitioner.

Variants

It will be appreciated that reference herein to KLF4 and other factors includes those embodiments described above, as well as sequence variants or fragments (e.g. protein fragments of at least 25, 50, 100, 150, 200, 250, 300, 350, 400, 450 or more amino acids in length) which retain the ability to direct the specific function of the factor, including for example induction of MET by KLF4.

For example, non-human variants may be used. Examples include variants of primate, rodent, porcine, bovine, canine, equine, feline origin.

Any such variants or fragments may be used in the methods of the present invention, for example, either in methods involving contacting the cells with KLF4 and/or other factors, or methods involving expressing KLF4 and/or other factors in the cells.

In a particular embodiment, the KLF4 used in the present invention may be obtained from cDNA found in Addgene plasmid 19770.

Polypeptides or peptides that have substantial identity to proteins encoded by the cDNA found in the Addgene plasmids or substantial identity to the representative amino acid sequences provided herein for KLF4 may also be used. Similarly, nucleotide sequences encoding any of these polypeptides, peptides or proteins, or nucleotide sequences having substantial identity thereto, may be used in the methods of the present invention.

Two sequences are considered to have substantial identity if, when optimally aligned (with gaps permitted), they share at least approximately 50% sequence identity, or if the sequences share defined functional motifs. In alternative embodiments, optimally aligned sequences may be considered to be substantially identical (i.e., to have substantial identity) if they share at least 60%, 70%, 75%, 80%, 85%, 90%, 95%, 96%, 97%, 98%, or 99% identity over a specified region. The term “identity” refers to sequence similarity between two polypeptides molecules. Identity can be determined by comparing each position in the aligned sequences.

A degree of identity between amino acid sequences is a function of the number of identical or matching amino acids at positions shared by the sequences, for example, over a specified region. Optimal alignment of sequences for comparisons of identity may be conducted using a variety of algorithms, as are known in the art, including the ClustalW program, available at http://clustalw.genome.ad.ip, the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85:2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). For example, the “BLAST 2 Sequences” tool, available through the National Center for Biotechnology Information (through the internet at http://www.ncbi.nlm.nih.gov/BLAST/bl2seq/wblast2.cqi) may be used, selecting the “blastp” program at the following default settings: expect threshold 10; word size 3; matrix BLOSUM 62; gap costs existence 11, extension 1. In another embodiment, the person skilled in the art can readily and properly align any given sequence and deduce sequence identity and/or homology by visual inspection.

Methods of Inhibition

Inhibition of ARX expression in the context of the present invention may use small inhibitory RNAs (siRNAs). ARX gene expression can be reduced by contacting the cell with a small double stranded RNA (dsRNA), or a vector or construct causing the production of a small double stranded RNA, such that ARX gene expression is specifically inhibited (i.e. RNA interference or RNAi). Methods for selecting an appropriate dsRNA or dsRNA-encoding vector are well known in the art for genes whose sequence is known (e.g. U.S. Pat. Nos. 6,573,099 and 6,506,559; and International Patent Publication Nos. WO 01/36646, WO 99/32619, and WO 01/68836).

Antisense oligonucleotide constructs can also function as inhibitors of ARX gene expression for use in the present invention. Anti-sense oligonucleotides, including anti-sense RNA molecules and anti-sense DNA molecules, would act to directly block the translation of ARX mRNA by binding thereto and thus preventing protein translation or increasing mRNA degradation, thus decreasing the level of ARX protein, and thus activity, in a cell. For example, antisense oligonucleotides of at least 10 consecutive bases from the sequence, more preferably at least 15 (e.g. at least 20, 25) bases and complementary to unique regions of the mRNA transcript sequence encoding ARX can be synthesized and administered, e.g., by conventional phosphodiester techniques. Perfect complementarily between the sequence of the antisense molecule and that of the target gene or messenger RNA is not required, but is generally preferred. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

Ribozymes can also function as inhibitors of ARX gene expression for use in the present invention. Ribozymes are enzymatic RNA molecules capable of catalyzing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyze endonucleolytic cleavage of ARX mRNA sequences are thereby useful within the scope of the present invention. Specific ribozyme cleavage sites within any potential RNA target are initially identified by scanning the target molecule for ribozyme cleavage sites, which typically include the following sequences, GUA, GuU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays. Both antisense oligonucleotides, siRNAs and ribozymes useful as inhibitors of ARX gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-0-methyl rather than phosphodiesterase linkages within the oligonucleotide backbone.

Expression Vectors

Where the methods involve expressing the differentiation factors (e.g. KLF4) in the cell, this may involve transfecting or transducing the cell with nucleic acids encoding the differentiation factors.

Generally speaking, those skilled in the art are well able to construct vectors and design protocols for recombinant gene expression. Suitable vectors can be chosen or constructed, containing, in addition to the elements of the invention described above, appropriate regulatory sequences, including promoter sequences, terminator fragments, polyadenylation sequences, marker genes and other sequences as appropriate. For further details see, for example, Molecular Cloning: a Laboratory Manual: 2nd edition, Sambrook et al, 1989, Cold Spring Harbor Laboratory Press or Current Protocols in Molecular Biology, Second Edition, Ausubel et al. eds., John Wiley & Sons, (1995, and periodic supplements).

Expression of the factors may involve expression from an expression vector, in particular a mammalian expression vector. The expression vector may be of any suitable structure which provides expression of the factors. As will be appreciated, a suitable promoter will be operably linked to the coding region for the particular factor. For example, a coding sequence is operably linked to a promoter if the promoter activates the transcription of the coding sequence. Preferably the transcription factor is KLF4.

Suitable expression systems are well known in the art and do not per se form part of the present invention. Particular example nucleic acid delivery systems are summarised in WO2012/006440.

Vectors include but are not limited to, plasmids, cosmids, DNA or RNA viruses (bacteriophage, animal viruses, and plant viruses), and artificial chromosomes (e.g., YACs), such as retroviral vectors (e.g. derived from Moloney murine leukemia virus vectors (MoMLV), MSCV, SFFV, MPSV, SNV etc), lentiviral vectors (e.g. derived from HIV-1, HIV-2, SIV, BIV, FIV etc.), adenoviral (Ad) vectors including replication competent, replication deficient and gutless forms thereof, adeno-associated viral (AAV) vectors, simian virus 40 (SV-40) vectors, bovine papilloma virus vectors, Epstein-Barr virus vectors, herpes virus vectors, vaccinia virus vectors, Harvey murine sarcoma virus vectors, murine mammary tumor virus vectors, Rous sarcoma virus vectors. Preferred viruses which can be used to generate viral vectors are retroviruses (Miller et al., Am. J. Clin. Oncol., 15(3):216-221, 1992) and lentiviruses. Lentiviral vectors are well known in the art (see, for example, Naldini et al, Science, 272(5259):263-267, 1996; Zufierey et al., Nat. Biotechnol., 15(9):871-875, 1997; Blomer et al., J. Virol, 71(9): 6641-6649, 1997; U.S. Pat. Nos. 6,013,516 and 5,994,136). Lentiviral vectors are a special type of retroviral vector which are typically characterized by having a long incubation period for infection. Furthermore, lentiviral vectors can infect non-dividing cells. Lentiviral vectors are based on the nucleic acid backbone of a virus from the lentiviral family of viruses.

Typically, a lentiviral vector contains the 5′ and 3′ LTR regions of a lentivirus, such as SIV and HIV. Lentiviral vectors also typically contain the Rev Responsive Element (RRE) of a lentivirus, such as SIV and HIV. Examples of lentiviral vectors include those of Dull, T. et al., “A Third-generation lentivirus vector with a conditional packaging system” J. Virol 72(11):8463-71 (1998);

For example, an adenovirus vector may be used to carry cDNA of human KLF. An exemplary vector is Addgene plasmid 19770 (ad-KLF4). It will be understood that the transcription factors may be co-expressed from one or more expression vectors.

Aspects of the invention described herein may be used with the conditions, cells, factors and methods described in GB Patent Application (GB1408558.3; Attorney Reference: SMK/GB6968556). The content of GB1408558.3 is incorporated herein by cross-reference. In particular the examples and experimental data shown in GB1408558.3 are incorporated herein by reference.

Aspects of the invention described herein may be used with the conditions, cells, factors and methods described in GB Patent Application (Attorney Reference: SMK/GB6996508) that was filed on the same day as the present application. The content of GB Patent Application (Attorney Reference: SMK/GB6996508) is incorporated herein by cross-reference. In particular the examples and experimental data shown in GB Patent Application (Attorney Reference: SMK/GB6996508) are incorporated herein by reference.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. Islet enriched pancreatic cells form fibroblast-like monolayers and dedifferentiate in adherent cell culture.

A: Phase contrast images taken in culture from day 0 to passage 6. QRT PCR analysis of endocrine, epithelial, mesenchymal (B) and pluripotency markers (C) in cells harvested from passage 1 to passage 9 in tissue culture. Data are presented expressed relative to glyceraldehyde 3-phosphate dehydrogenase.

FIG. 2. KLF4 overexpression induces morphological change with up-regulation of epithelial markers and down-regulation of mesenchymal markers.

Islet enriched pancreatic cell clusters were cultured in RPMI with 10% FBS and allowed to adhere and expand. At passage 6 (Day 0) cells were transduced with Ad-KLF4 and compared with Ad-EGFP treated cells. A: Phase-contrast images showing morphological changes at time points post infection with Ad-KLF4 or Ad-EGFP. The cells changed morphology over time becoming more epithelial and less fibroblast-like in appearance when compared to non-transduced cells (UTR). B: Cells were harvested at time points for gene expression by QRT/PCR. Data were expressed relative to glyceraldehyde-3-phosphate dehydrogenase and 60S ribosomal protein L13a (n=3). A two-way ANOVA was performed with Bonferroni post hoc test comparing treatment groups with Ad-EGFP. For all analyses, *P<0.05 **P<0.01 ***P<0.001. Transduction of IEF-derived MSCs with Ad-KLF4 results in an increase in epithelial markers E-cadherin and Ep-Cam and decrease in MSC markers vimentin, α-smooth muscle actin and SNAIL2 with increase in ZEB1. Similar data was obtained with Ad-GFP as a control (data not shown). This suggests that Ad-KLF4 is stimulating a mesenchymal to epithelial transition (MET). C: Immunocytochemical staining of epithelial marker E-cadherin and mesenchymal marker vimentin at day 4 post transduction with Ad-KLF4 versus control. Nuclei were counterstained with DAPI. Scale bar=20 μm.

FIG. 3. KLF4 overexpression induces re-expression of both endocrine and exocrine markers.

Islet enriched pancreatic cell clusters were cultured in RPMI with 10% FBS and allowed to adhere and expand. At passage 6 cells were transduced with Ad-KLF4 or Ad-EGFP. A. Cells were harvested at time points for gene expression which was expressed relative to glyceraldehyde-3-phosphate dehydrogenase and 60S ribosomal protein L13a (n=3) and expressed as mean±SEM. Samples were also fixed at day 4 for immunocytochemistry. B: Staining for Amylase and CK19. C. Staining for C-peptode and E-cadherin or Chromogranin A and E-cadherin. Nuclei were counterstained with DAPI. Scale bar=20 μm. D: Temporal gene expression of pluripotency markers. A two-way ANOVA was performed on all QRT PCR analyses with Bonferroni post hoc test comparing treatment groups with NA. For all analyses, P*<0.05 **P<0.01 ***P<0.001.

FIG. 4. Effect of KLF4 is transient in Lenti-KLF4 treated cells and induces apoptosis in Ad-KLF4 treated cells

Dedifferentiated islet enriched pancreatic cells at passage 6 were transduced with lenti-KLF4-GFP (KLF4) vs. lenti emGFP (emGFP) and harvested at time points. A: Temporal gene expression of key pancreatic and epithelial markers. Cells were harvested at time points for gene expression which was expressed relative to glyceraldehyde-3-phosphate dehydrogenase and 60S ribosomal protein L13a and expressed as mean±SEM (n=3). A two-way ANOVA was performed on all QRT PCR analyses with Bonferroni post hoc test comparing treatment groups with lenti-GFP. For all analyses, P*<0.05 **P<0.01 ***P<0.001. B. Cleaved caspase 3 (CASP3) co-expresses with E-cadherin (ECAD) in Ad-KLF4 but not Ad-EGFP treated cells. Nuclei were counterstained with DAPI. Scale bar=20 μm. C. Cells were transduced with Ad-KLF4 or Ad-EGFP and fixed at day 4. A TUNEL assay was performed followed by counterstaining with DAPI. >1500 nuclei were counted per treatment and cells identified as apoptotic calculated as a percentage of all cells.

FIG. 5. Suspension culture enhances the effects of Ad-KLF4

Dedifferentiated islet enriched pancreatic cells at passage 6 were transduced with Ad-KLF4 or Ad-EGFP KLF4 and cultured overnight in adherent cell culture conditions. The cells were then either left in adherent conditions or detached with accutase and transferred to suspension for a further 4 days. A: Phase contrast comparison of treatments at day 5 in culture. B: Samples harvested for QRT PCR and gene expression analysis for epithelial and mesenchymal markers which were expressed relative to glyceraldehyde-3-phosphate dehydrogenase and 60S ribosomal protein L13a and expressed as mean±SEM (n=3). A one-way ANOVA was performed on all QRT PCR analyses with Dunnett post hoc test comparing treatment groups vs. Ad-EGFP control. Unpaired t-tests were performed where necessary. For all analyses, P*<0.05 **P<0.01 ***P<0.001. The anomalous effect of Ad-GFP on GCG has not been reproducible.

FIG. 6—β-cell derived and acinar cell derived MSCs can be differentiated towards adipocyte and osteoblast lineages.

Lineage traced acinar and beta derived cells were expanded in culture for 6 weeks. The expanded dsRed⁺ MSCs were FACS sorted and cultured for a further 2 months. (A) Morphology of sorted dsRed⁺ cells counterstained with DAPI. Sorted acinar (AMY-dsRED) and β-cell derived (IND-dsRed) MSCs were cultured for 10 days on a commercially available adipocyte differentiation cocktail and stained for lipid droplets (LipidTox) (B), or cultured for 14 days on a commercially available osteocyte differentiation cocktail and stained for osteocalcin.

FIG. 7. Ad-KLF4 induces INS-dsRed MSCs to differentiate down both endocrine and exocrine lineages.

A FACS sorted and expanded INS-dsRED MSCs were transduced with Ad-KLF4 or Ad-EGFP and samples fixed for immunocytochemistry and stained for E-cadherin, chromogranin A and CK19. Nuclei were counterstained with DAPI. Scale bar=20 μm. B Samples were harvested for QRT PCR and gene expression expressed relative to relative to glyceraldehyde-3-phosphate dehydrogenase and 60S ribosomal protein L13a and expressed as mean±SEM (n=3). Unpaired t-tests were performed between Ad-KLF4 and Ad-EGFP transduced cells. For all analyses, P*<0.05 **P<0.01 ***P<0.001.

FIG. 8. Ad-KLF4 induces AMY-dsRed MSCs to differentiate down both endocrine and exocrine lineages.

A FACS sorted and expanded AMY-dsRED MSCs were transduced with Ad-KLF4 or Ad-EGFP and samples fixed for immunocytochemistry and stained for amylase, chromogranin A and CK19. Nuclei were counterstained with DAPI. Scale bar=20 μm. B Samples were harvested for QRT PCR and gene expression expressed relative to relative to glyceraldehyde-3-phosphate dehydrogenase and 60S ribosomal protein L13a and expressed as mean±SEM (n=3). Unpaired t-tests were performed between Ad-KLF4 and Ad-EGFP transduced cells. For all analyses, P*<0.05 **P<0.01 ***P<0.001.

FIG. 9. Expression of endogenous KLF4 in untreated IEF-derived MSCs cells and exogenous KLF4 in cells transduced with Ad-KLF4. Expression levels of the exogenous KLF4 peak at D2 with a subsequent decrease to D10. Cells were transduced with or without Ad-Klf4 and fixed at day 4 for immunocytochemistry. A: Klf4 staining in untransduced cells (NA) (FITC exposure time=1978 ms). B: Klf4 Staining following Ad-Klf4 treatment (FITC exposure time 473 ms). C: Mouse Klf4 gene expression at time points in culture post transduction relative to glyceraldehyde-3-phosphate dehydrogenase and 60S ribosomal protein L13a (n=3). D: Klf4 and E-cadherin (ECAD) staining at different FITC exposure times 4 days post transduction. Scale bar=20 μm.

FIG. 10. E-cadherin (ECAD) positive cells are more widespread following ad-KLF4 treatment in the presence of serum.

FIG. 11. Rho-kinase inhibition does not enhance redifferentiation. A. Islet enriched pancreatic cells were treated with ad-Klf4 with and without Rho-associated protein kinase inhibitor Y27632 (20 uM). Samples were harvested at up to 10 days post infection and gene expression measured relative to glyceraldehyde-3-phosphate dehydrogenase and 60S ribosomal protein L13a (n=3). A one-way ANOVA was performed with Dunnett post hoc test comparing treatment groups. For all analyses, *P<0.05 **P<0.01 ***P<0.001. B: Samples were fixed and permeabilised at day 6 for E-cadherin and cleaved caspase-3.

FIG. 12. Laminin isoforms 511 and 522 enhance attachment of MSCs derived from islet derived MSCs became fully attached to enriched pancreatic cells but do not facilitate redifferentiation. A: Glass coverslips were pre-coated overnight with laminin isoforms LN111, LN211, LN332, LN411, LN421, LN511, and LN521 followed by the addition of islet derived MSCs. Phase contrast images were captured at 8 hr after plating cells and 4 days post infection with ad-Klf4. Scale bar=100 um. B: Coverslips were fixed at day 4 and stained for E-cadherin (ECAD), C-peptide (CPEP) and Cleaved Caspase-3. Representative data is shown for LN521. Arrows mark cleaved Caspase-3 staining. Scale bar=20 μm. C: Cells transduced with Ad-Klf4 on laminin isoforms were harvested for QRT-PCR at 4 days for expression of insulin (INS), amylase (AMY), CK19 and ECAD. Data are shown relative to glyceraldehyde 3-phosphate dehydrogenase and 60s ribosomal protein L13a and expressed as mean SEM (n=3). A one-way ANOVA was performed with Dunnett post hoc test comparing treatment groups with no additions (NA). For all analyses, *P<0.05 **P<0.01 ***P<0.001.

FIG. 13. Passaged pancreatic MSCs are plated in tissue culture dishes and transduced with KLF4+ the reprogramming transcription factors. One day after transduction the cells are placed into suspension culture and cultured for another 8 days in the presence of betacellulin, exendin-4 and nicotinamide. At day 6 the suspension cultures are transduced with siARX. During the 8 days glucose concentration is kept at 2.5 mM.

FIG. 14. KLF4 expression drops more rapidly than eGFP expression.

FIG. 15. Protocol for overcoming apoptosis. The protocol use in Example 2 is illustrated. This represent a preferred suspension culture protocol.

FIG. 16. Suspension culture enhances MET and endocrine and exocrine gene expression are maintained.

FIG. 17. Representative electron microscopic images of cells reprogrammed with siArx. Unlike non reprogrammed cells, reprogrammed cells are rich in dense secretory granules (A). Scale bar=2 μm. High magnification images (B and C) of dense core vesicles with different morphologies in reprogrammed cells. Scale bar=0.5 μm (B) and 0.1 μm (C).

FIG. 18. RT-qPCR analysis of the three main endocrine hormones insulin (INS), glucagon (GCG) and somatostatin (SST) and the transcription factors PDX1, PAX4, MAFA, NEUROD, NGN3 and NKX6.1 in untreated (N/A) or cells reprogrammed (siARX) in the absence or presence of ZnCl₂ (10 μM). Expression was normalised to glyceraldehyde 3-phosphate dehydrogenase. Data are representative of triplicate experiments and represented as mean+/−standard error of the mean.

FIG. 19. C-peptide ELISA measurements of cell extracts from untreated cells (N/A) or cells reprogrammed (siARX) in the absence or presence of ZnCl₂ (10 μM). C-peptide levels were expressed level to protein content and represent 3±SD (n=3). ***p<0.001 relative to NA and **p<0.01 relative to siARX.

### EXAMPLES

### SUMMARY

Human islet enriched pancreatic cells were cultured in RPMI with 10% FBS and allowed to dedifferentiate and expand. The resultant population of MSCs were transduced with an adenovirus containing KLF4 (ad-KLF4) and incubated for between 2 and 10 days. Gene expression was assessed by real-time quantitative PCR. Morphological changes and protein distribution were assessed by immunocytochemistry.

Treatment with ad-KLF4 resulted in re-expression of epithelial genes E-Cadherin and EPCAM. This was associated with reduced expression of mesenchymal markers vimentin, snai2 and α-SMA maximally at 48 hr post transduction (all p=<0.001). Markers of differentiated pancreatic cells were also up-regulated, including insulin by 891.2% (p=<0.0001), amylase by 1117.9% (p=0.002) and CK19 (p=0.002) by 3844%. Endocrine transcription factors NGN3, MafA, Nkx6.1 and NeuroD1 were all significantly up-regulated.

Cells staining for E-cadherin, insulin, amylase and CK19 were seen on fluorescence immunocytochemistry at 96 hr post ad-KLF4, but not in control cells. Genetic lineage tracing confirmed at least some of these cells were derived from beta cells. These findings hold promise that beta cells which have dedifferentiated and expanded ex-vivo can be redifferentiated by directly promoting an MET.

### Example 1

Materials and Methods

Culture of Human Islet Enriched Pancreatic Fractions

All human tissue was procured with appropriate ethical consent. Human islets were isolated from brain-dead adult donor pancreata at the Scottish Islet Isolation Laboratory (SNBTS, Edinburgh, UK) under GMP conditions. Islet-enriched fractions not used for human transplantation and exocrine-enriched fractions were transported to Aberdeen, where the cells were immediately plated at a density of 3×10⁵ clusters on 75 cm² tissue culture flasks (Greiner, Stonehouse, UK) and cultured in serum-containing medium (SCM) prepared using RPMI 1640 (Gibco, Life Technologies, Paisley, UK) supplemented with 10% foetal bovine serum (FBS), 10 mmol/L HEPES, 1 mmol/L sodium pyruvate (all from Gibco), and 75 mmol/L b-mercaptoethanol (Sigma Aldrich, Dorset, UK). Cells were passaged every 5-7 days using trypsin 0.05% and EDTA (0.02%: Gibco).

In experiments carried out using adherent cell culture, cells were seeded at 2.8×10⁴ cells per cm² with SCM switching to SCM without HEPES after overnight attachment. In experiments requiring suspension culture, cells were seeded at 3.13×10⁴ cells per cm² in ultra-low attachment plates (Corning) using the same cell density as adherent culture controls. Serum-free medium (SFM) was prepared with RPMI supplemented with 1% BSA and 10 ug/ml insulin, 5.5 ug/ml transferrin and 6.7 ng/ml sodium selenite.

Laminin isoforms LN111, LN211, LN332, LN411, LN421, LN511 and LN521 were obtained from Biolamina AB, Stockholm, Sweden.

Viral Vectors for KLF4

A plasmid encoding for mouse KLF4 was obtained through the Addgene plasmid repository (Addgene plasmid 19770, www.addgene.org). The plasmid was expanded using an E-coli vector and isolated and purified using Purelink™ HiPure Plasmid Filter Purification Kit (Invitrogen cat: 1147565). The plasmid was subsequently linearised using Pacl endonuclease and amplified using HEK293A cell line. Viral particles were extracted and titered before use. pAd-EGFP was also obtained from Addgene. Adenoviral-mediated transduction was performed in SFM at a multiplicity of infection of 25.

Human KLF4 cDNA was inserted into pLenti6 to generate Lv-CMV-hKLF4-IRES-hrGFP, with Lv-CMV-emGFP as a control.

Genetic Lineage Tracing

Genetic lineage tracing was performed as previously described (Lima et al., 2013).

Fluorescence Activated Cell Sorting

Cells for sorting were incubated in StemPro® Accutase® (Life Technologies, Paisley UK) for 10 min followed by pipetting to break up clusters. Cells were then passed through a 70 μm cell strainer and dsRed positive cells sorted on a BD Influx™ Cell Sorter using a phycoerythrin 593/40 filter. Collected dsRed positive cell fractions were then expanded in adherent culture for a further 8 weeks prior to further experiments.

Differentiation Towards Adipocyte and Osteocyte Lineages

Acinar and islet-derived dsRed sorted cells were seeded at a density of 2×10⁴ cells on 22×22 mm coverslips. The cells were cultured in the presence of StemPro Osteogenesis differentiation medium (Life Technologies) for 20 days or StemPro Adipogenesis medium (Life Technologies) for 10 days.

Quantitative RT-PCR

Total RNA was extracted using TRIzol® and treated with DNaseI (both Life Technologies) followed by cDNA synthesis using 1 μg of RNA per sample. qRT-PCR mixtures were prepared using SensiMix II probe kit (Bioline, London, UK) and TaqMan gene expression assays (Applied Biosystems, Paisley, UK) as per manufacturer's instructions. cDNA was amplified on Roche LightCycler 480® for 50 cycles. Samples were run in triplicate and normalised to glyceraldehyde 3-phosphate dehydrogenase (GAPDH) and/or 60S ribosomal protein L13a (RPL13a). Data was analysed using the 2^{-ΔΔ}CT method (Brodsky et al., 1999). Statistical analysis was performed using GraphPad Prism software and the Student t test or one-way/two-way ANOVA, followed by the Dunnett post hoc test, were used as appropriate.

TABLE 1
List of Taqman ® gene expression primers

| Gene | Assay ID |
|---|---|
| CDH1 | Hs01023894_m1 |
| EPCAM | Hs00901885_m1 |
| VIM | Hs00185584_m1 |
| SNAI2 | Hs00950344_m1 |
| ZEB1 | Hs00232783_m1 |
| ACTA2 | Hs00909449_m1 |
| GAPDH | Hs99999905_m1 |
| RPL13A | Hs04194366_g1 |
| INS | Hs00355773_m1 |
| GCG | Hs00174967_m1 |
| SST | Hs001174949_m1 |
| AMY2B | Hs00949916_m1 |
| KRT19 | Hs00761767_s1 |
| PDX1 | Hs00236830_m1 |
| NGN3 | Hs01875204_s1 |
| MAFA | Hs01651425_s1 |
| NKX6.1 | Hs00232355_m1 |
| OCT4 | Hs04260367_gH |
| SOX2 | Hs01053049_s1 |
| NANOG | Hs04260366_g1 |
| KLF4 (mouse) | Mm00516105_g1 |

Fluorescence Immunocytochemistry

Cells were cultured on 22×22 mm or 13 mm round glass coverslips and fixed with 4% paraformaldehyde in phosphate-buffered saline (PBS) followed by permeabilisation in ice-cold methanol. Cells were washed thrice in PBS and blocked with 10% goat serum (Gibco) in tris-buffered saline Triton X-100 for 1 hr. Cells were incubated with the relevant primary antibody overnight at 4° C., washed in PBS, then incubated with secondary antibody goat Alexa Fluor® 488 F(ab′)₂ goat anti-rabbit IgG (H+L) or Alexa Fluor® 594 goat anti-mouse IgG (H+L) at dilution 1:400. Coverslips were then washed and mounted on slides with VECTASHIELD® HardSet mounting medium with DAPI. Fluorescent Images were captured using a Zeiss Axio Imager.M2 and collated with AxioVision software. Antibodies used are shown in Table 2.

TABLE 2
antibodies used in immunofluorescence

| Antigen | Antibody host | Source | Dilution used |
|---|---|---|---|
| E Cadherin | Mouse | Becton Dickinson | 1:200 |
| Vimentin | Rabbit | Dako | 1:200 |
| | Mouse | Proteintech | 1:200 |
| Amylase | Rabbit | Sigma | 1:100 |
| NGN3 | Rabbit | Abcam | 1:200 |
| KLF4 | Rabbit | Millipore | 1:300 |
| C-peptide | Mouse | Cell Signalling | 1:1000 |
| Oct4 | Rabbit | Abcam | 1:500 |
| Cleaved Caspase-3 | Rabbit | NE biolabs | 1:200 |
| Osteocalcin | Mouse | Abcam | 1:50 |

TUNEL Assay

Cells were seeded on 22×22 mm coverslips and after 24 h infected with Ad-KLF4. Cell exposed to UV light for 1 h were used as positive control. A terminal deoxynucleotidyl transferase mediated dUTP nick-end labelling (TUNEL) assay was performed using the ApopTag Fluorescein Direct In Situ Apoptosis Detection kit (Millipore, Watford, UK) according to manufacturer's instructions. Cell counts were performed over 10 randomly selected fields over 2 slides with at least 700 nuclei per slide. TUNEL positive cells were identified using the FITC channel on a Zeiss Axio Imager.M2 fluorescence microscope.

siRNA Based Knockdown.

Knockdown of Arx in transdifferentiating cells was performed by transfection with a pool of specific targeting small inhibitory RNAs, or scrambled controls (Dharmacon, Loughborough, UK). 100 nM siRNA was transfected on day 6 of the transdifferentiation protocol using Dharmafect 1 (Dharmacon), according to the manufacturer's instructions.

Results

Freshly Isolated Islet-Enriched Pancreatic Cells Undergo EMT in Adherent Cell Culture

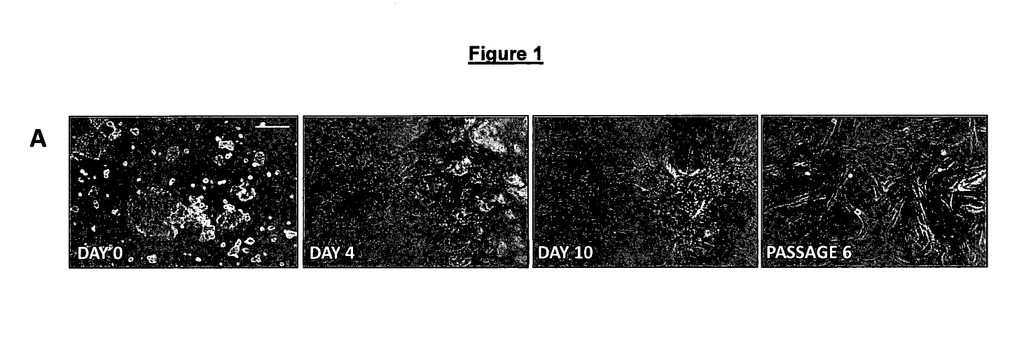

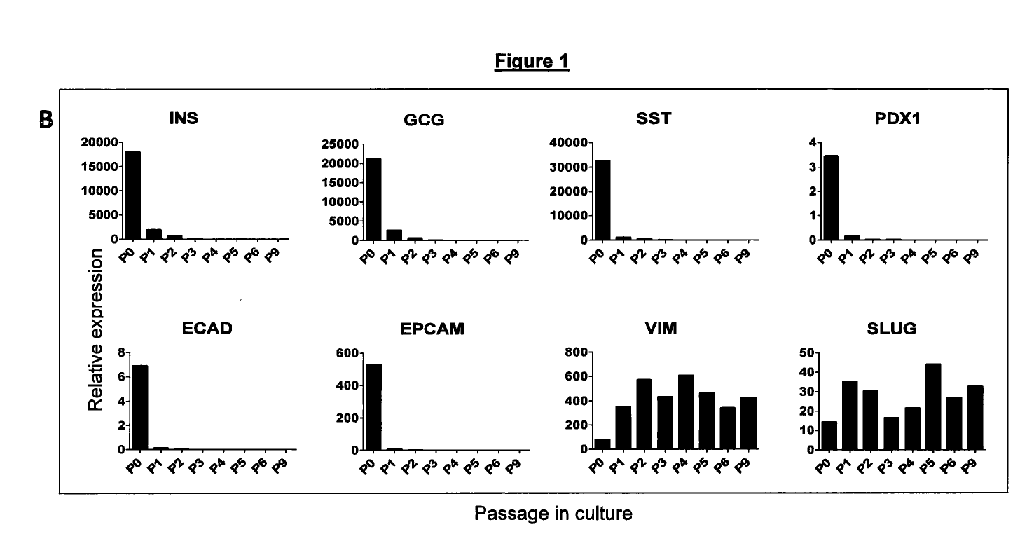

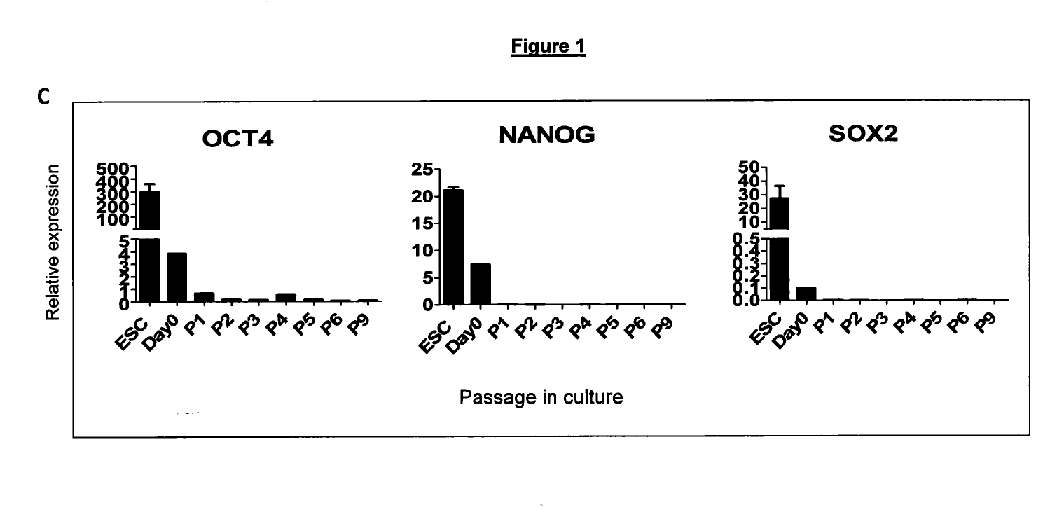

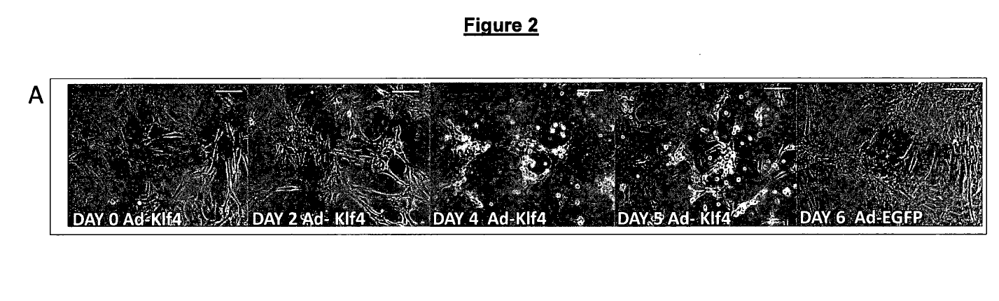

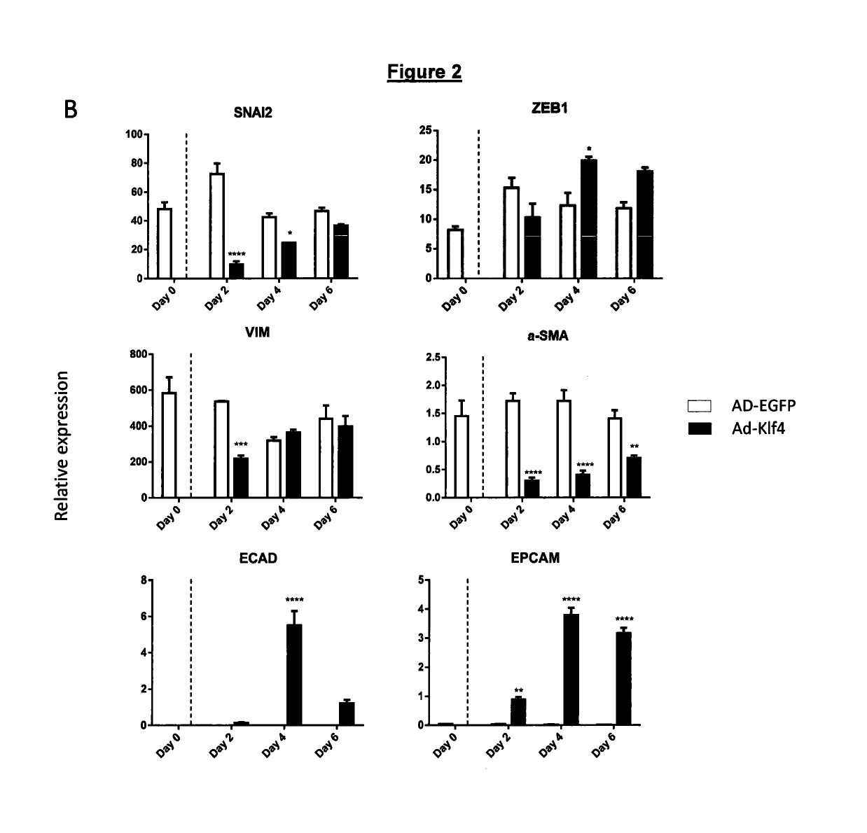

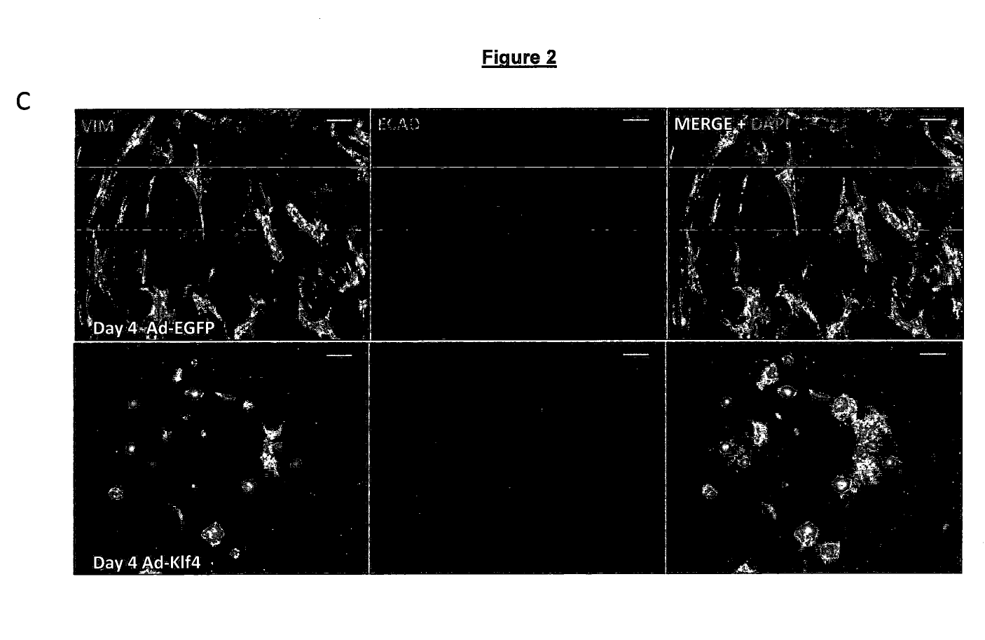

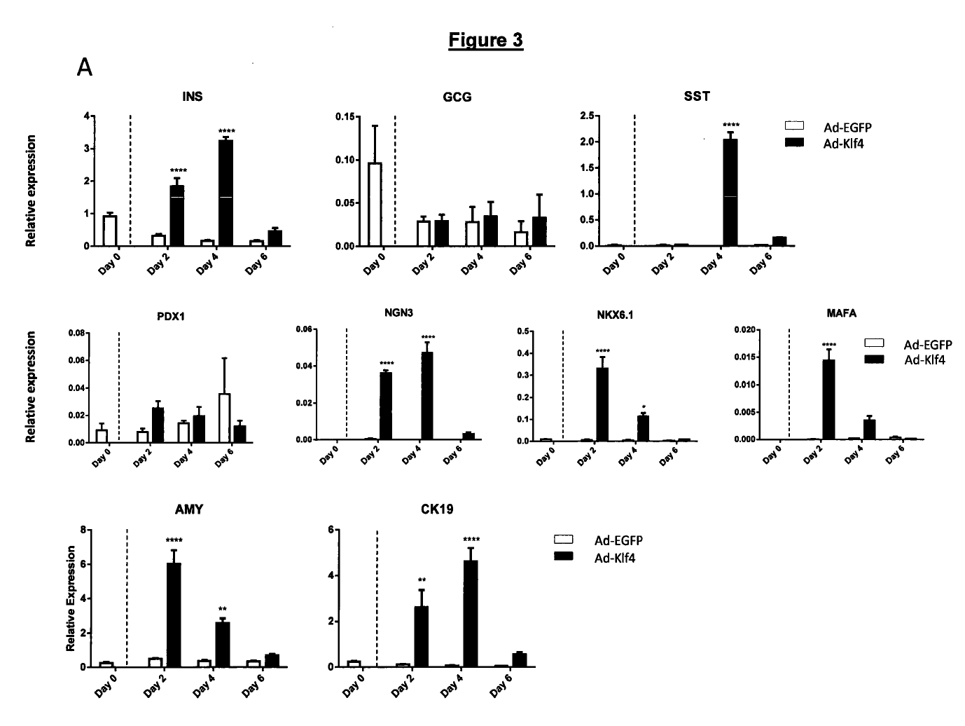

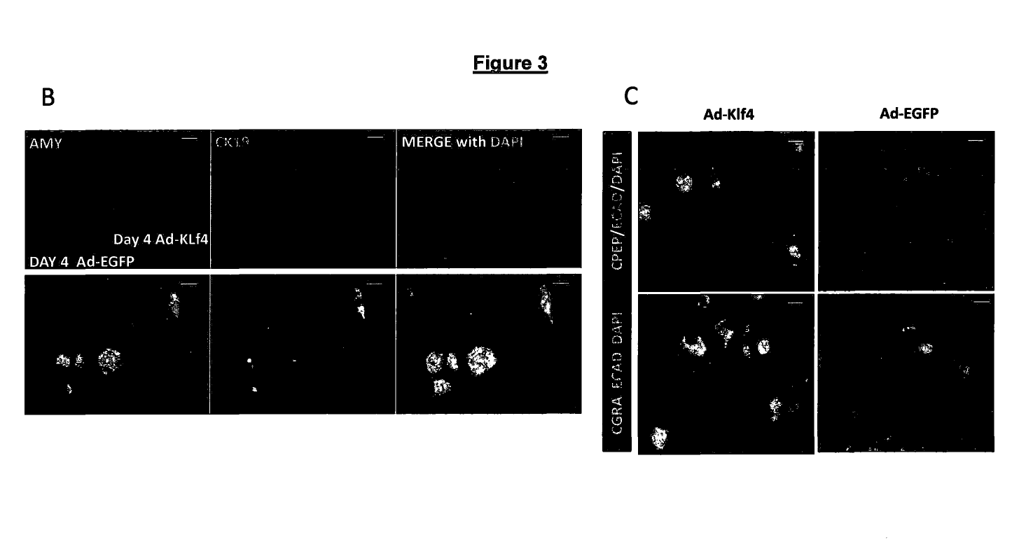

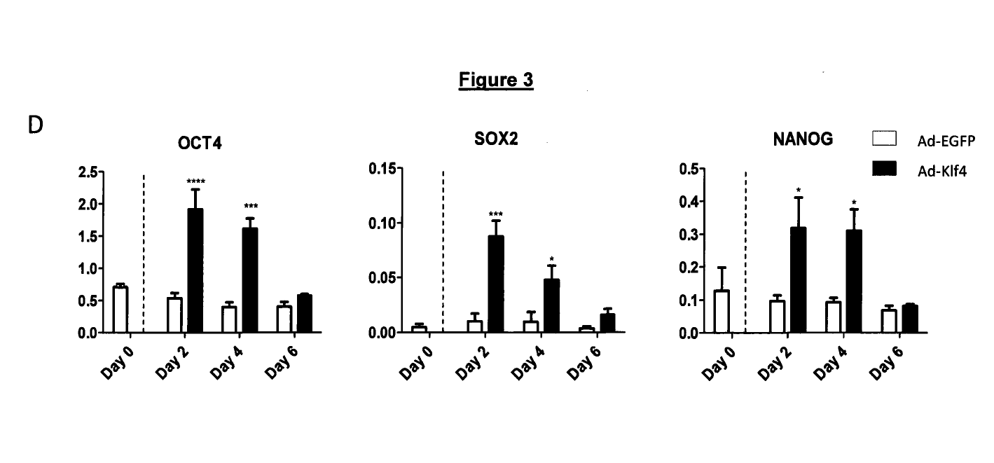

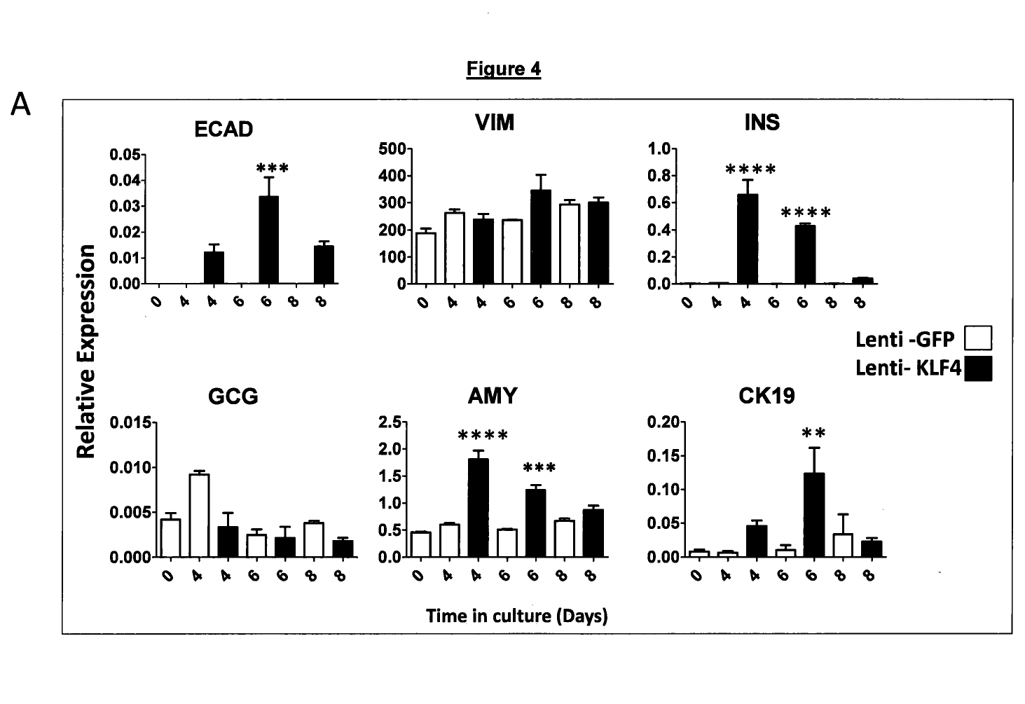

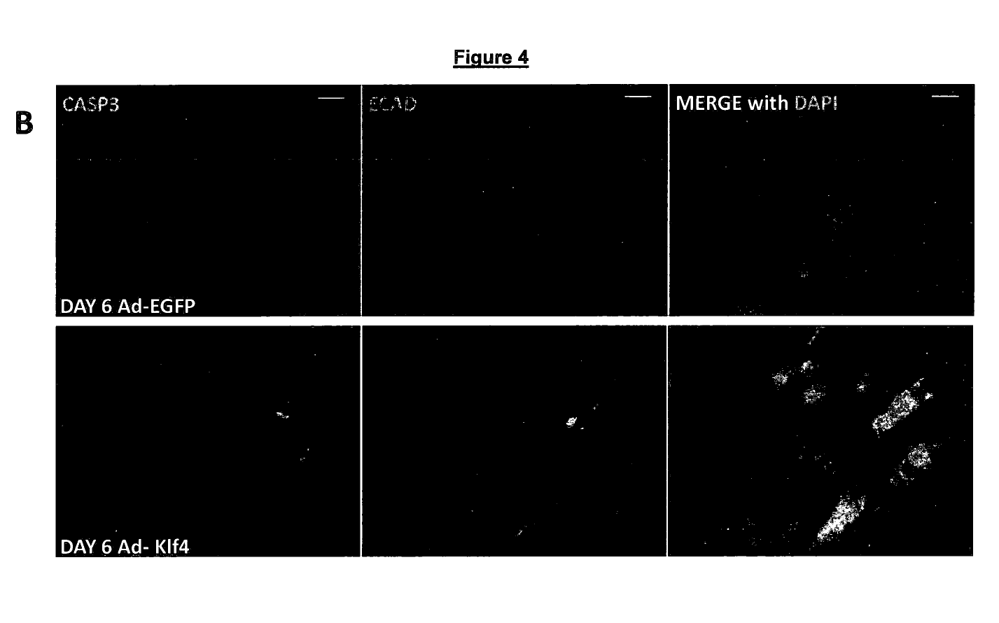

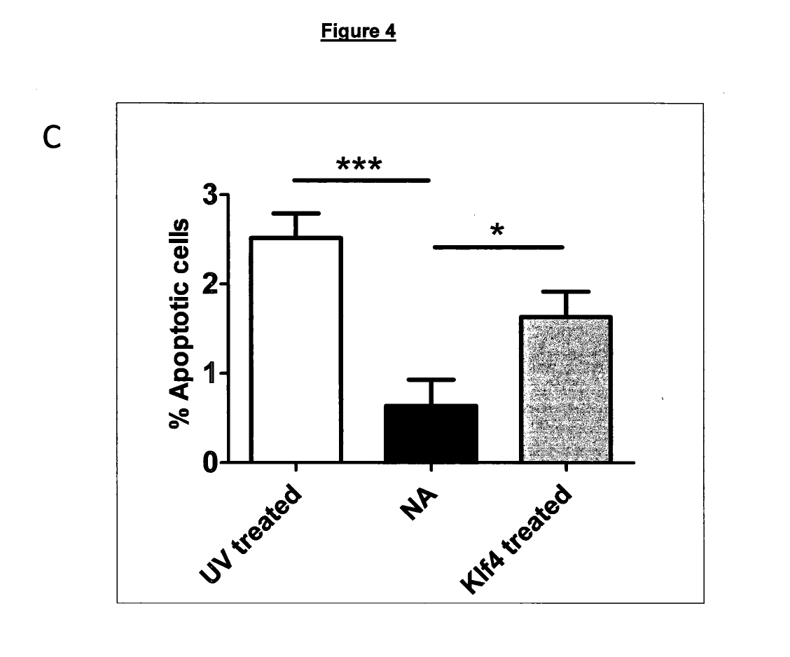

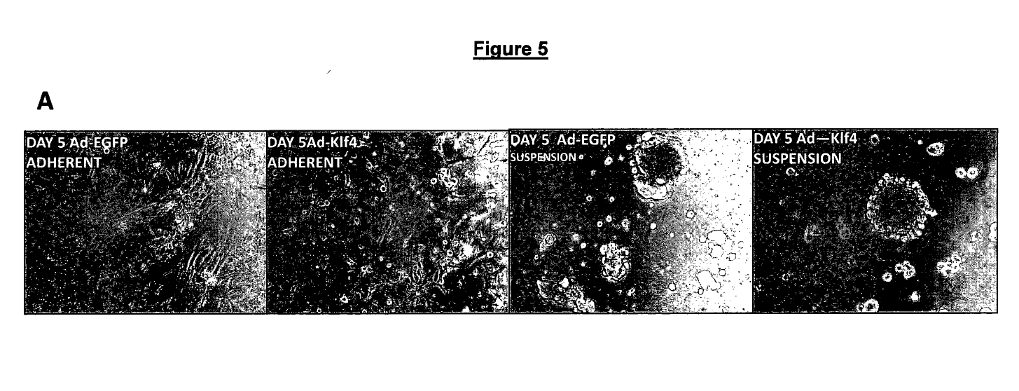

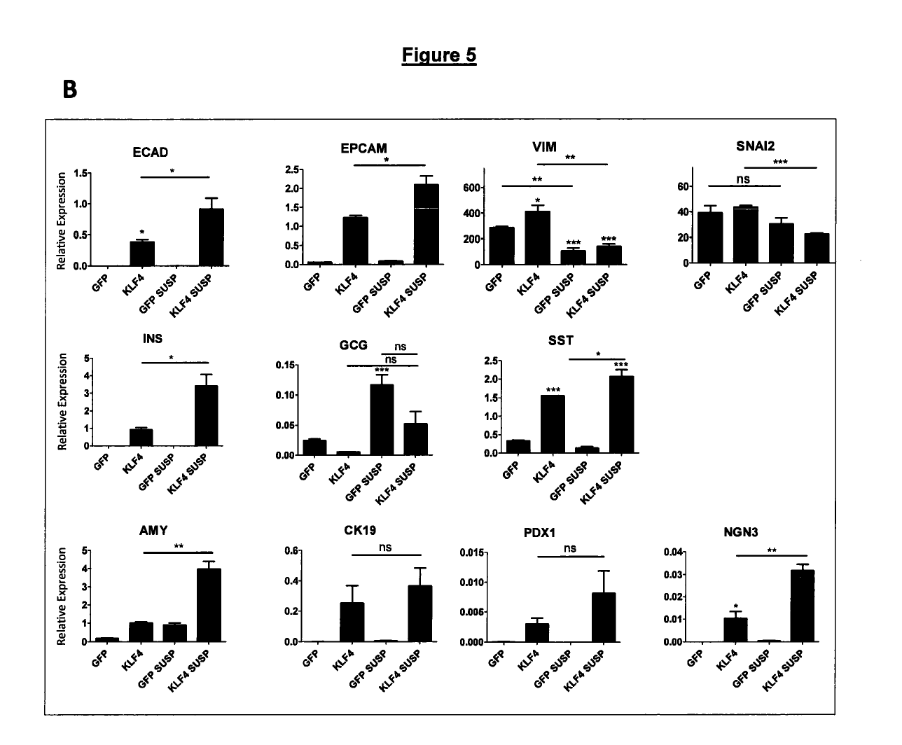

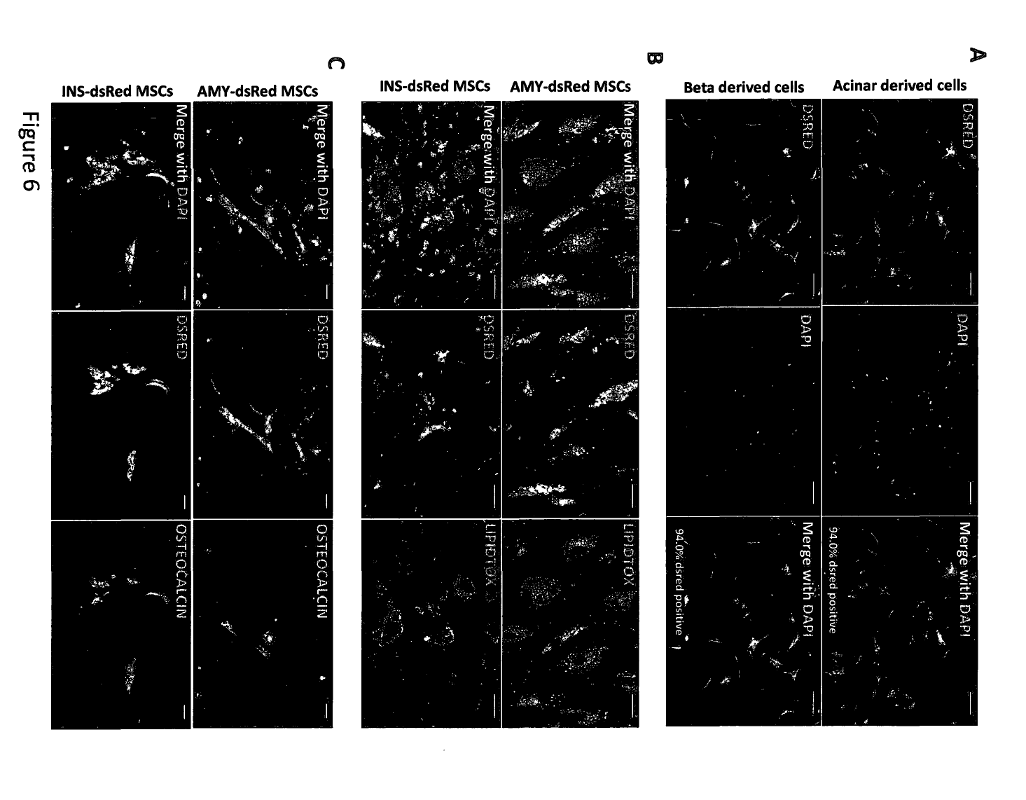

Islet-enriched pancreatic cells (IEPCs) used in experiments were designated at 83% islet purity by the isolation facility and were composed of epithelial-like clusters prior to culture. Dithizone staining confirmed this high level of purity (FIG. 1A).

When plated in plastic culture dishes the islet clusters attached to the dish. Within 24 h fibroblast-like cells started to migrate out of the cluster, forming a proliferative monolayer that spread throughout the culture dish (FIG. 1A) (Gallo et al., 2007). This monolayer could be repeatedly passaged.

Early in adherent cell culture, C-peptide positive cells were widespread and co-stained with epithelial marker E-cadherin, but not with vimentin, although vimentin positive cells were present within the islet. At day 10 in culture, C-peptide and glucagon stained cells were infrequent and co-stained with vimentin in the case of glucagon, but not C-peptide (FIG. 1B). At passage 6 in culture (approximately 4 weeks), the cell population resembled a monolayer of mesenchymal stromal cells (FIG. 1A). The presence of other cell types in early cell culture including acinar cells, ductal cells and MSCs were noted by staining for amylase, CK19 and vimentin respectively (data not shown).

In keeping with previous studies (Beattie et al., 1997; Gershengorn et al., 2004) there was a rapid decrease in pancreatic and epithelial markers (insulin, glucagon, somatostatin, PDX1, E-cadherin and EpCAM) with a concomitant increase in mesenchymal markers (vimentin and SNAI2 (SLUG)) with time in culture (FIG. 1B). This was in keeping with the view that cells had undergone rapid dedifferentiation.

We have previously shown that these fibroblast-like cells express surface antigens that are characteristic of mesenchymal stromal cells (MSCs), and in keeping with the properties of MSCs can be differentiated into adipocytes, osteoblasts and chondrocytes (Lima et al., 2013).

Genetic lineage tracing confirmed that this mesenchymal monolayer was derived in part from epithelial to mesenchymal transitioning (EMT) of insulin expressing β-cells (FIG. 6).

Although we could detect weak expression (relative to ES cells) of pluripotency markers (OCT4, SOX2 and NANOG) in the newly plated islets, this was rapidly lost as the cells underwent EMT, and there was no transient increase around passage 5 (FIG. 10) as reported by others (White et al., 2011).

KLF4 Overexpression in Adherent Culture Induces an MET and Redifferentiation Towards Pancreatic Cell Types

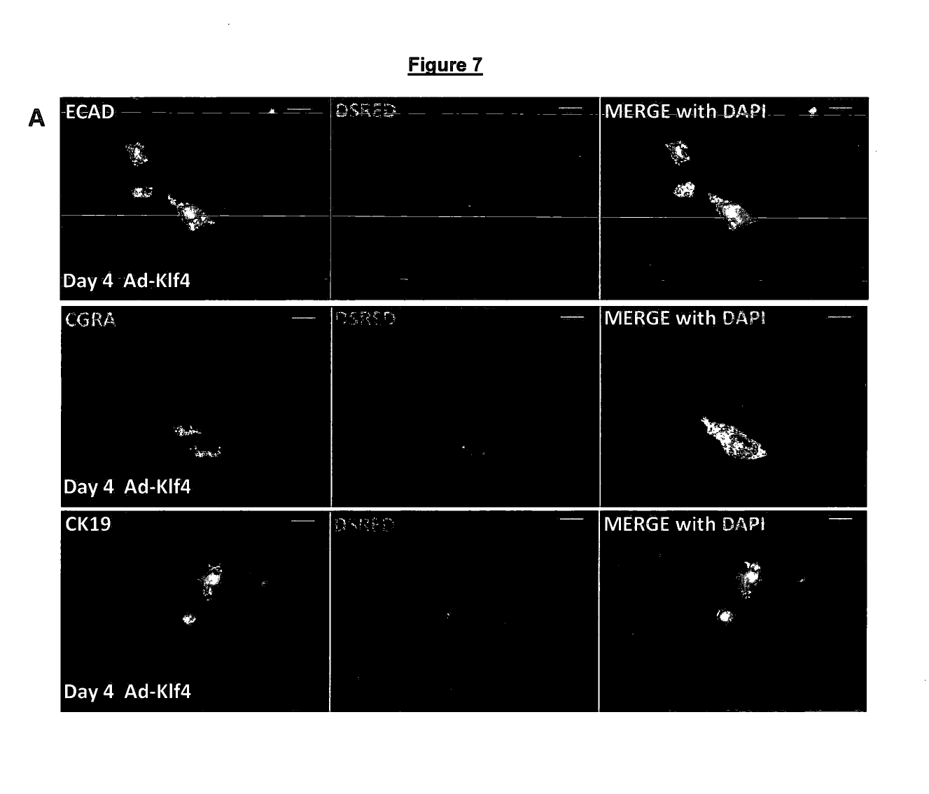

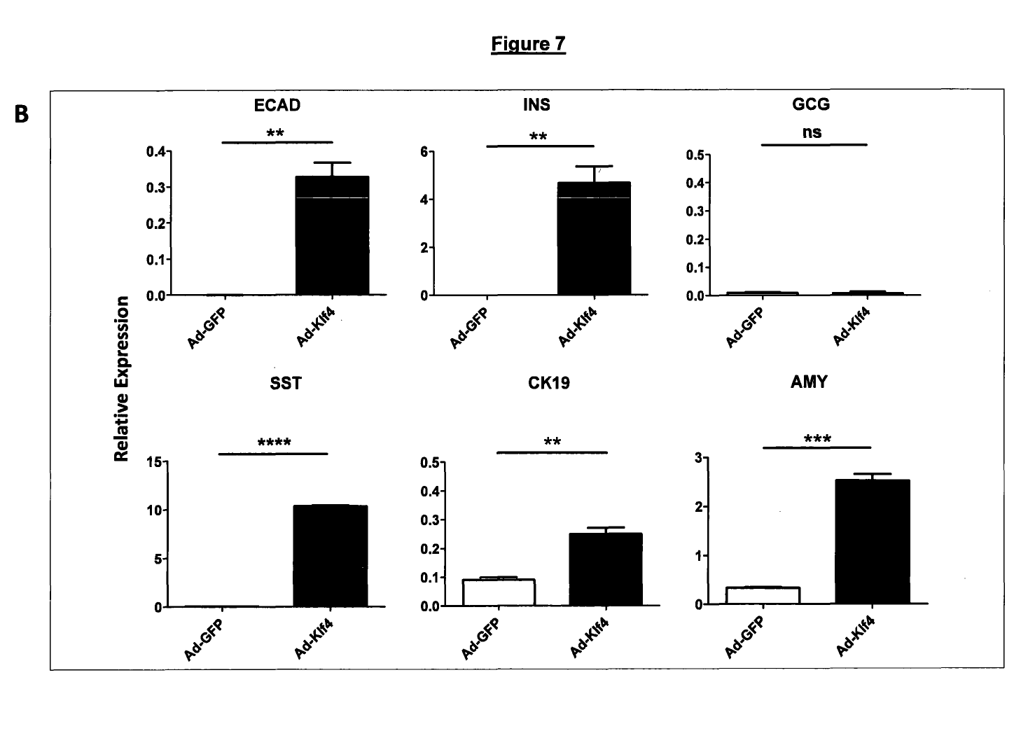

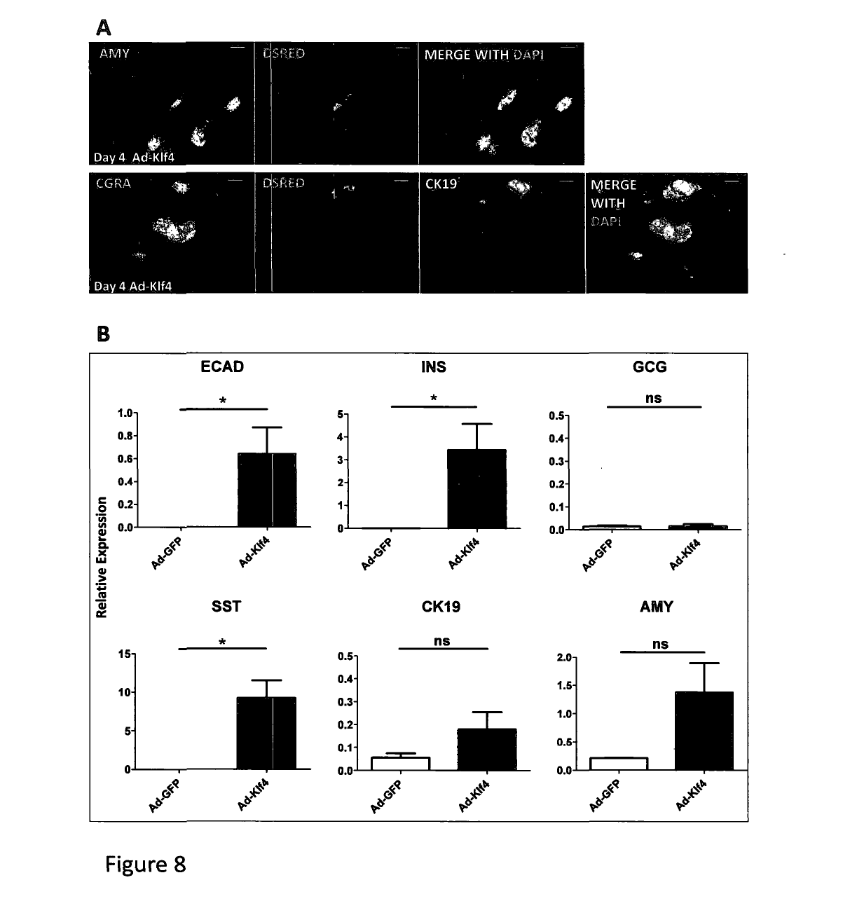

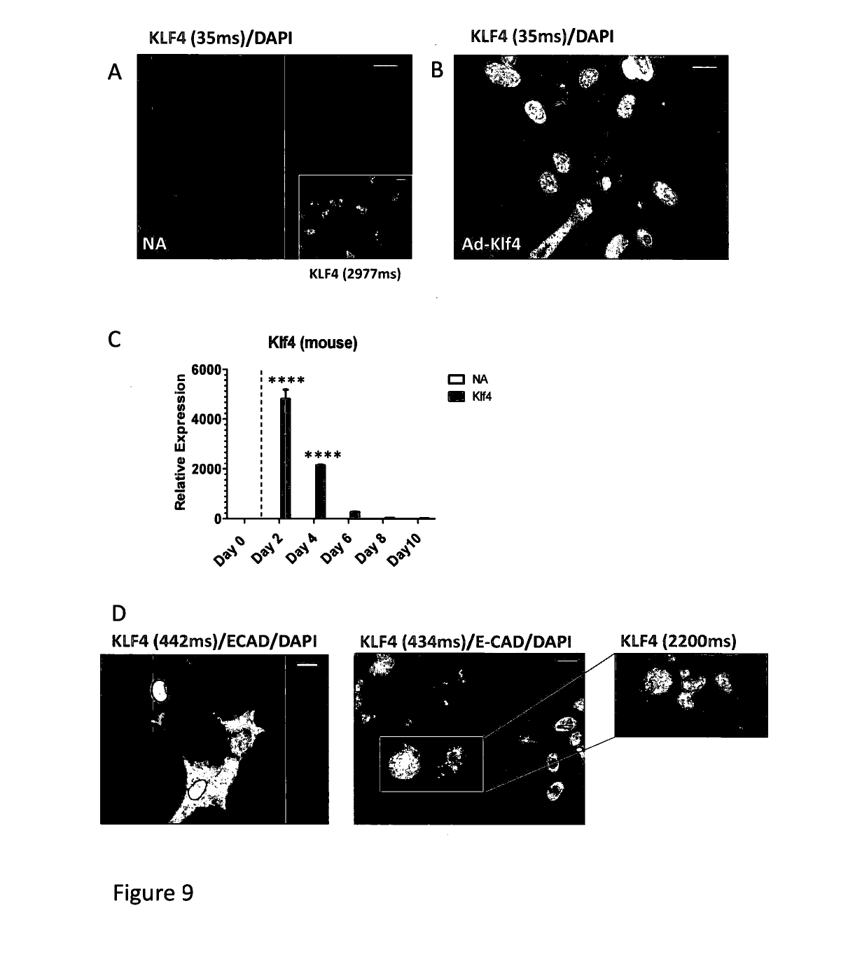

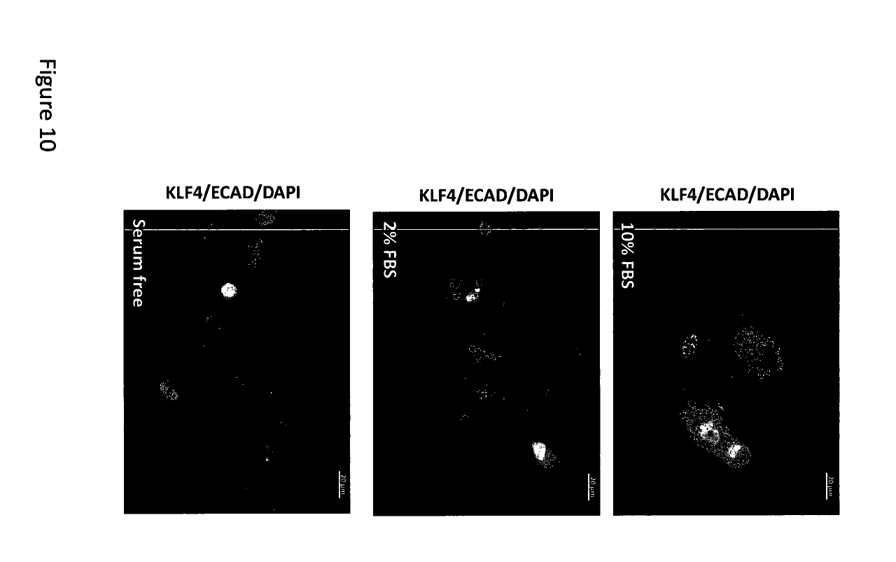

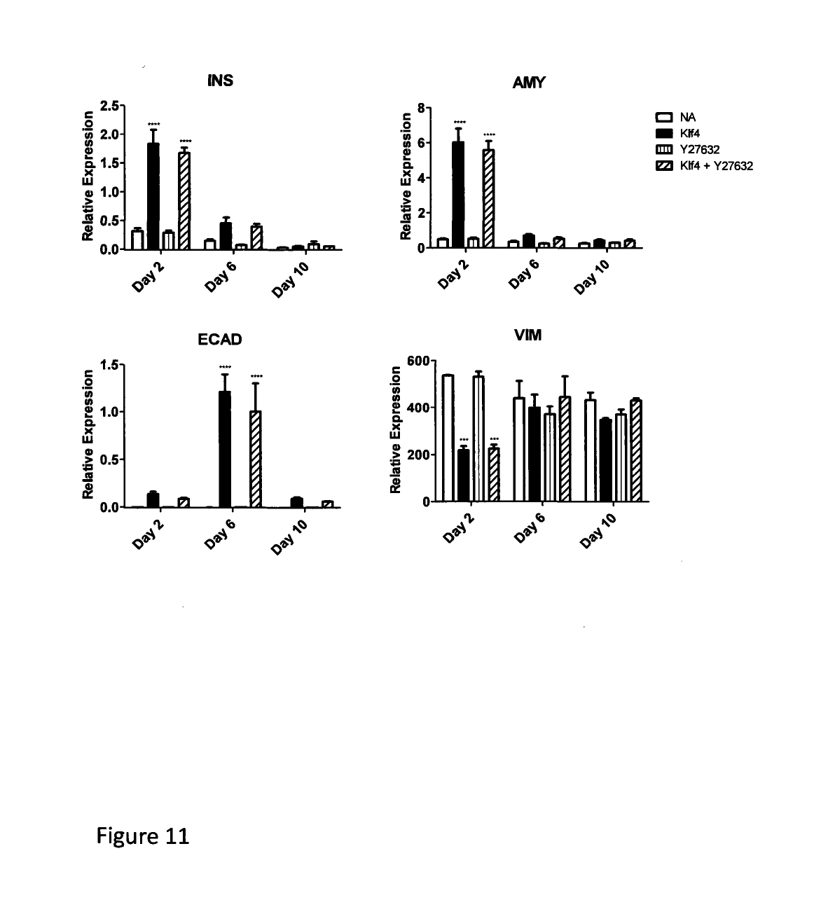

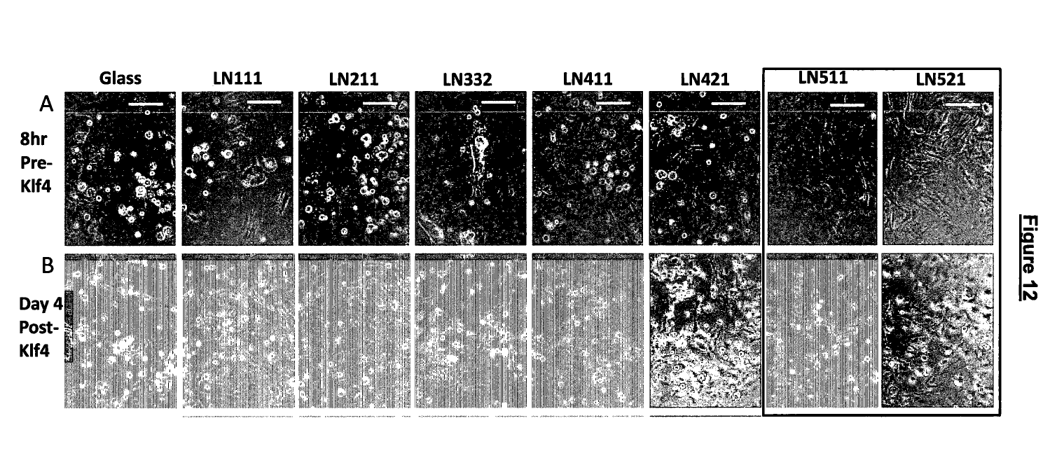

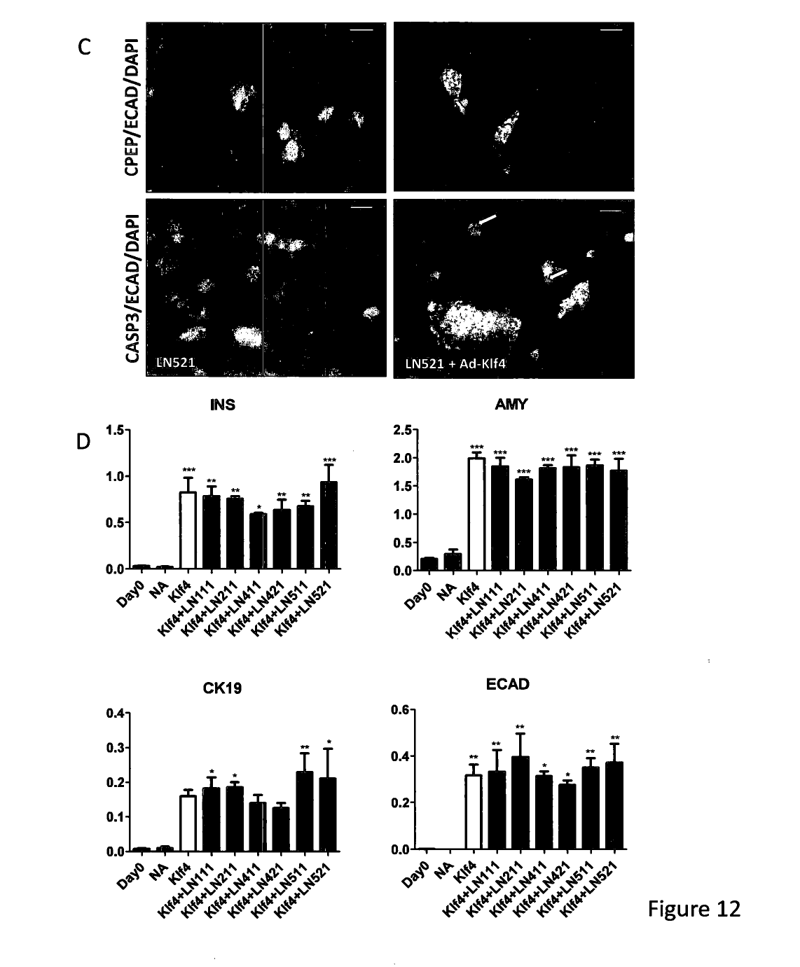

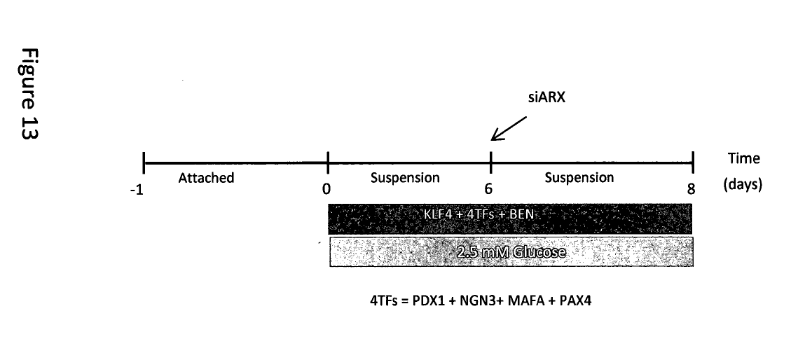

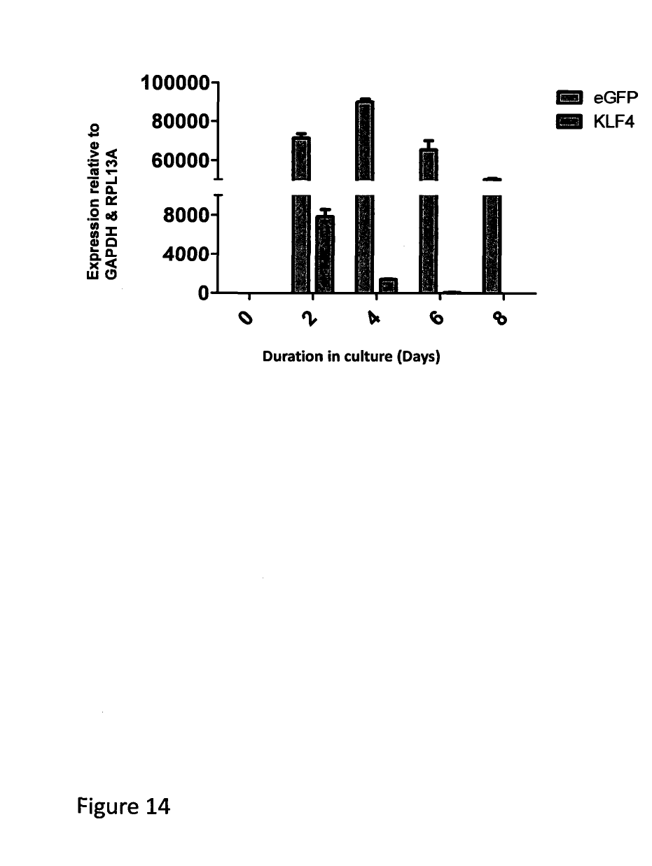

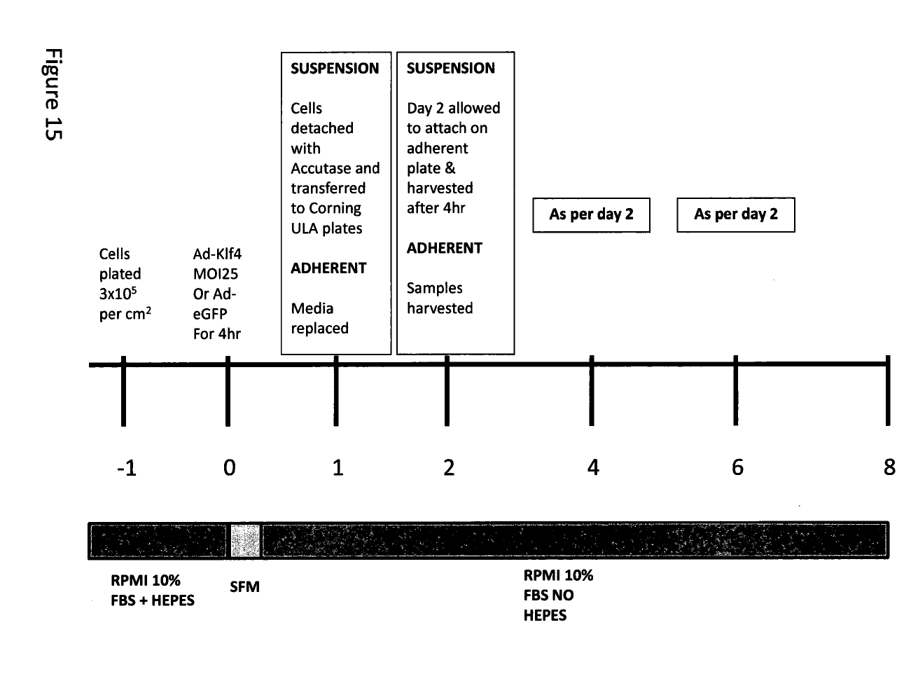

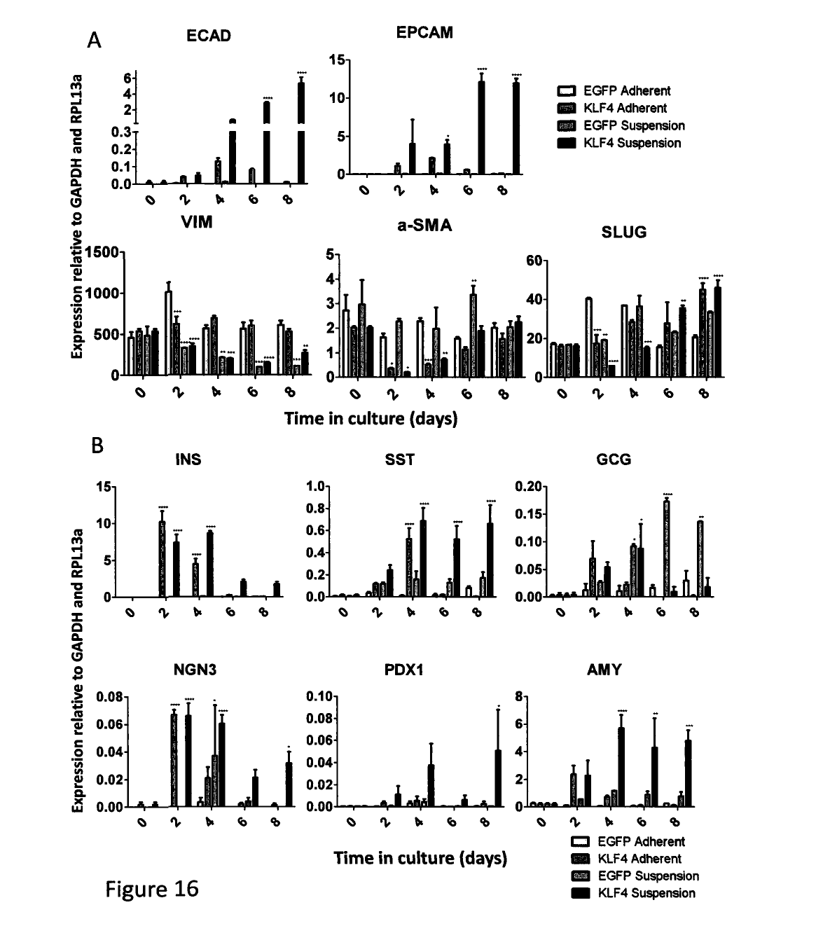

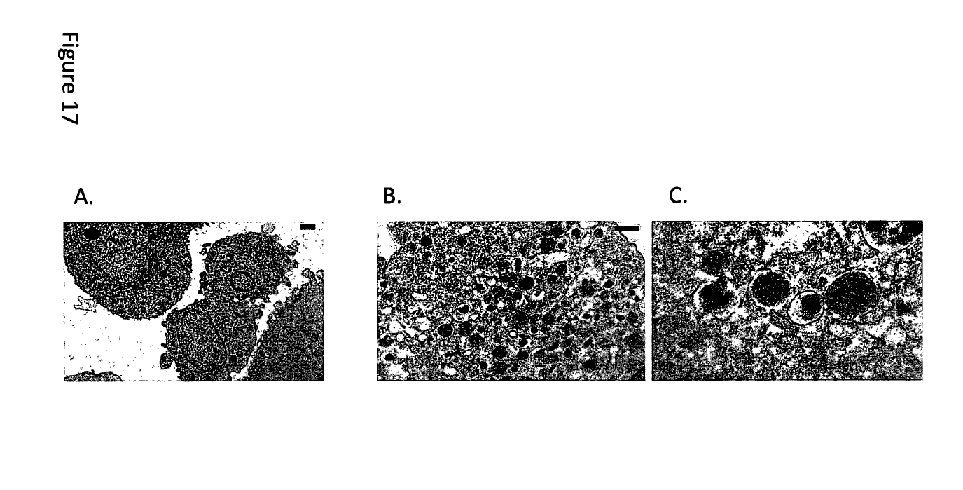

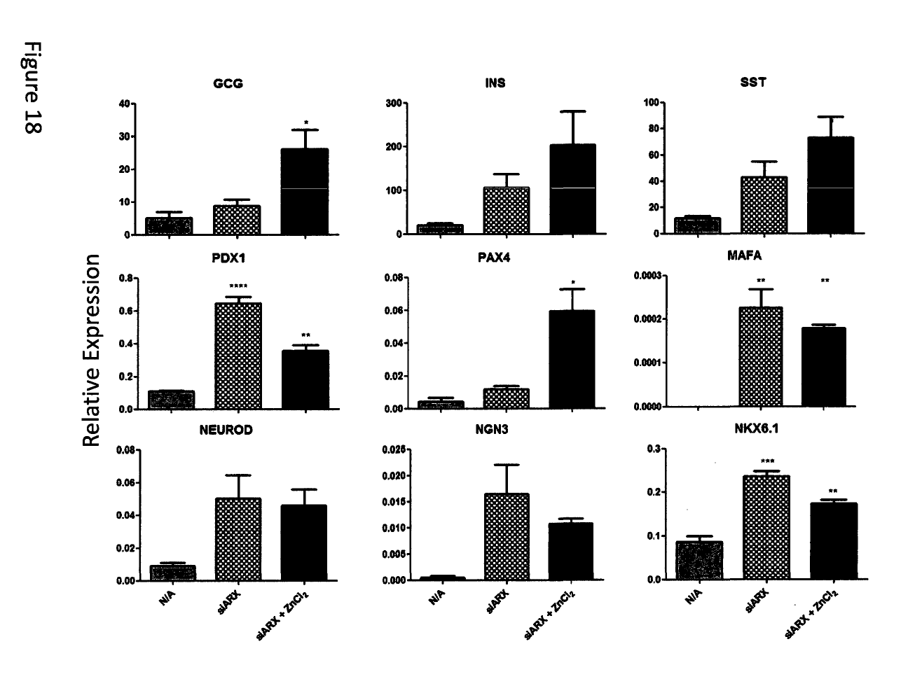

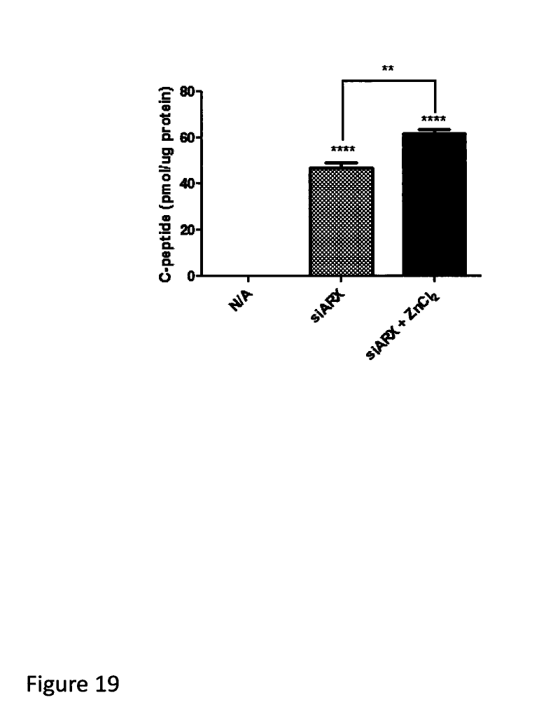

Islet derived MSCs displayed low levels of nuclear KLF4 staining as evidenced by immunocytochemistry (FIG. 9A). Ad-KLF4 was efficiently taken up by islet derived MSCs (FIG. 9B) with rapid up-regulation of mouse specific KLF4 gene expression peaking at day 2, followed by a subsequent fall to undetectable levels by day 8 (FIG. 9C). In the presence of serum, Ad-KLF4 induced significant morphological changes with aggregation and many cells transitioning towards a more rounded epithelial form (FIG. 2A). Gene expression of epithelial markers E-cadherin (ECAD) and epithelial cell adhesion molecule (EPCAM) were rapidly upregulated to significant levels peaking at day 4 with a subsequent decrease towards day 6 (FIG. 2B). E-cadherin presence was shown to be widespread in Ad-KLF4 transduced cells by immunocytochemistry, with positive cells displaying a more epithelial morphology (FIG. 2C).

Conversely gene expression of mesenchymal markers vimentin and α-SMA, and transcriptional repressor Snai2 were downregulated significantly at day 2 followed by a rise towards baseline upon further culture (FIG. 2D). This was accompanied by perinuclear relocation of vimentin in cells staining positive for E-cadherin (FIG. 2C). In contrast to the findings of earlier studies (Gershengorn et al., 2004), omitting serum from the media significantly reduced the number of E-cadherin positive cells (FIG. 10). In summary these data are consistent with the occurrence of an MET taking place in response to Ad-KLF4 treatment.

In addition to the upregulation of epithelial markers, KLF4 overexpression led to a significant increase in the expression of endocrine hormones insulin and somatostatin (FIG. 3A) and pancreatic transcription factors (PDXI , NGN3, NKX6.1 and MAFA) that are present in developing and mature beta cells (FIG. 3A). Interestingly, there was no increase in expression of glucagon (FIG. 3A).

Expression levels of the acinar marker amylase and ductal marker CK19 were also significantly increased (FIG. 3B). C-peptide positive cells were infrequently observed by immunocytochemistry; however chromogranin A, a pan-endocrine marker was seen throughout following Ad-KLF4 treatment (FIG. 3C). Immunocytochemistry also revealed widespread staining for amylase and CK19 with many cells staining for both in Ad-KLF4 treated but not untreated cells. Similar co-expression of amylase and CK19 was observed during the dedifferentiation of exocrine enriched cells (Houbracken et al., 2011; Lima et al., 2013). Ad-KLF4 also stimulated a transient increase in pluripotency factors OCT4, NANOG and SOX2 (FIG. 3C). This is not unexpected since KLF4 regulates NANOG expression (Chan et al., 2009; Zhang et al., 2010), while SOX2 and OCT4 repress expression of the mesenchymal markers SNAII and Vimentin. These findings are in keeping with KLF4 induced redifferentiation of IEF-derived MSCs towards pancreatic cell types.

The transient nature of the KLF4 effect could be attributed in part to the use of non-integrating adenoviral vectors. To address this we created a lentiviral vector overexpressing human KLF4, which would integrate into the host genome. Lenti-KLF4 induced an increase in E-cadherin, insulin, amylase and CK19, but not glucagon, expression. However, as seen with the Ad-KLF4 construct, the increased expression of these markers was transient (FIG. 4A). Collectively, these data suggest that exogenous KLF4 is capable of initiating a process of MET but that other factors might be required for further maturation and stabilisation of the epithelial phenotype. Some evidence in favour of the requirement for these factors was provided by the observed co-staining of the apoptotic marker CASP3 and E-cad in Ad-KLF4 infected cells (FIG. 4B), while a TUNEL assay, which measured a later stage apoptosis, revealed a significantly higher number of apoptotic cells following treatment with Ad-KLF4 (FIG. 4C).

Promoting Cell-to-Cell Contact in Suspension Culture Enhances KLF4 Induced Redifferentiation

We next hypothesised that adjusting the cell culture environment to promote survival of newly formed epithelial cells would enhance redifferentiation. Initial experiments involved treatment with Ad-KLF4 along with the Rho-associated kinase inhibitor (ROCK) Y27632, which has previously been effective in preventing apoptosis in dissociated pluripotent stem cells (Ohgushi et al., 2010) and suppressing pancreatic exocrine cell dedifferentiation (Lima et al., 2013) (Budd et al., 1993). However, no significant difference in gene expression was observed between treatment groups (FIG. 11), so Rho-kinase inhibition does not enhance redifferentiation.

We next investigated whether coating the culture dish with different laminin isoforms, including those known to interact with β-cells in the human basal lamina (Banerjee et al., 2012), would enhance Ad-KLF4-mediated redifferentiation. Freshly-plated islet derived MSCs became fully attached to laminin isoforms LN511 and LN521 after only 8 hours (FIG. 12A), while attachment to other isoforms and to glass took significantly longer (a full 24 hours). Four days after Ad-KLF4 transduction, superior attachment was observed on the LN521 coating, but not on the other isoforms (FIG. 12B). However, none of the laminin isoforms enhanced Ad-KLF4 expression of insulin, amylase, CK19 and E-cadherin (FIGS. 12C and 12D).

It has been previously shown that suspension culture in serum free media can enhance redifferentiation of islet- and exocrine-derived MSCs (Gershengorn et al., 2004; Rooman et al., 2000). Culture in suspension for 5 days led to the formation of epithelial-like clusters (FIG. 5A), but had no detectable effect on the expression of epithelial or mesenchymal markers (FIG. 5B). In monolayer culture Ad-KLF4 increased expression of epithelial markers (ECAD; EPCAM), and this effect was considerably enhanced when the Ad-Klf4 treated cells were subsequently placed in suspension, under which conditions a marked decrease in mesenchymal markers (vimentin and SNAI2 (SLUG)) was also observed (FIG. 5B).

Ad-KLF4 mediated expression of insulin, somatostatin, Pdx1, NGN3, amylase and CK19 were all enhanced in suspension culture (FIG. 5B). Ad-KLF4 had no effect on glucagon expression in suspension culture (FIG. 5).

These results suggest that suspension culture enhances the ability of Ad-KLF4 to induce MET and redifferentiation towards endocrine and exocrine pancreatic lineages. However, as seen in the cells that remained attached to the dish, the effect of Ad-KLF4 was transient.

β-Cell and Acinar Cell Derived MSCs Redifferentiate Down Both Endocrine and Exocrine Lineages Following Treatment with Ad-KLF4

The ability to induce islet-derived MSCs to undergo an MET, albeit transiently, gave us the opportunity to ask important questions regarding the redifferentiation potential of islet and acinar-derived MSCs. To map the origins of the MSC population, genetic lineage tracing was undertaken using an adenovirus containing Cre-recombinase under the control of the insulin or amylase promoters and a lentiviral vector containing a CMV driven dsRed reporter preceded by a floxed stop cassette blocking its expression (Houbracken et al., 2011; Lima et al., 2013). The cells were allowed to dedifferentiate, and after several passages the dsRed positive cells were sorted by flow cytometry and expanded to provide almost homogeneous (>94%) populations of MSCs (FIG. 6A) that were derived from either insulin positive β-cells (INS-dsRed MSCs) or amylase-positive acinar cells (AMY-dsRed MSCs). We were then able to demonstrate for the first time that MSCs derived from β-cells and acinar cells could be induced to differentiate down adipocyte and osteoblast lineages (FIGS. 6B and 6C). Furthermore, Ad-KLF4 could induce INS-dsRed and AMY-dsRed MSCs to express E-cadherin, insulin, somatostatin, CK19 and amylase (but not glucagon) with equal efficiency (FIGS. 7 and 8). These results indicate that β-cell and acinar cell derived MSCs have the ability to differentiate towards both endocrine and exocrine lineages after long term culture.

Low passage cells may redifferentiate more efficiently.

Treatment with Transcription Factors may Stabilise the Beta-Cell Phenotype upon the Action of KLF4

Passaged pancreatic MSCs are plated in tissue culture dishes and transduced with KLF4 and the reprogramming transcription factors PDX1, MAFA, NGN3 and PAX4. One day after transduction the cells are placed into suspension culture and cultured for another 8 days in the presence of betacellulin, exendin-4 and nicotinamide (BEN). At day 6 the suspension cultures are transduced with siARX. During the 8 days glucose is kept at 2.5 mM.

### Example 2

KLF4 Expression Drops More Rapidly than eGFP

Expression of KLF4 relative to GDDPH and RPL13A was measured after treatment with Ad-KL4 as described above. KLF4 expression was compared to expression of an eGFP control that was also introduced by adenovirus.

FIG. 14 shows that expression of KLF4 drops more rapidly than the eGFP control. This demonstrates that the transient expression of KLF4 was not due to the method of delivery (Adenovirus). It is more likely that KLF4 has a specific negative effect on the cells perhaps by stimulating apoptosis.

Culture Conditions (FIG. 15)

Cells were plated at a density of 3×10⁵ per cm² and cultured for one day in RPMI supplemented with 10% foetal bovine serum (FBS) and HEPES. After 24 h, the cells were incubated for in serum free medium (SFM). The cells were incubated for 4 h with the adenoviruses encoding KLF4 or eGFP at a multiplicity of infection (MOI) of 25. Serum Free medium was replaced with RPMI supplemented with 10% foetal bovine serum (FBS) and HEPES.

Suspension Culture—1 day after infection with adenovirus the cells were detached with

Accutase and transferred to Corning ULA plates. Cells were attached to adherent plates for 4 h before harvesting on days 2, 4, 6, and 8 after infection.

Adherent Culture—1 day after infection the media was replaced. Samples were harvested on days 2, 4, 6 and 8 after infection.

Suspension Culture Enhances MET

Suspension culture was used to promote cell aggregation and formation of belt forming junctions. Suspension culture overcame the transient effect of KLF4 on MET, particularly with respect to the epithelial markers which continued to rise. Vimentin remained at reduced levels for the duration of the time course. (FIG. 16A).

Endocrine and Exocrine Gene Expression was Maintained

Expression of both endogenous exocrine and endocrine factors after KLF-4 introduction was measured. Expression of these factors was maintained in suspension culture as compared to adherent culture (FIG. 16B).

### Example 3

Methods

Reprogramming of Human Exocrine Pancreatic Fractions

Human exocrine fractions were thawed and plated on tissue culture 9 cm² dishes (Greiner, Stonehouse, UK) and cultured for two days in RPMI 1640 (Gibco, Life Technologies) supplemented with 10% foetal bovine serum (FBS), 10 mM HEPES, 1 mM sodium pyruvate (all from Gibco) and 75 μM β-mercaptoethanol (Sigma Aldrich). After 48 h, the cells were incubated for another 72 h in serum free medium (SFM) prepared with RPMI 1640, insulin-transferrin-selenium (Gibco) and 1% bovine serum albumin (Sigma), supplemented with 10 μM SB431542, 2 μM Y27632, 1 μM 5-Aza-2′deoxycytidine and 10 mM sodium butyrate (all from Sigma). On the next day the cells were incubated for 4 h with the adenoviruses encoding pancreatic transcription factors PDX1, MAFA, NGN3 and PAX4. On the following day the medium was changed for SFM supplemented with 1 nM betacellulin (R&D systems, Abingdon, UK), 10 nM exendin-4 and 10 mM nicotinamide (both from Sigma). The medium was changed every two days for another 6 days before harvesting.

Knockdown of ARX was performed by transfection with a pool of specific targeting small inhibitory RNAs, or scrambled controls (all from Dharmacon, Loughborough, UK). siRNA (100 nM) transfected on day 6 of the reprogramming protocol using Dharmafect 1 (Dharmacon), according to the manufacturer's instructions.

ZnCl₂ was used at a concentration of 10 μM and was used in combination with the reprogramming adenoviruses and the siARX.

Transmission Electron Microscopy

Cells were detached from plates using Accutase™ (BD Biosciences, Oxford, UK) and subsequently fixed in 2.5% glutaraldehyde in 0.1M sodium cacodylate buffer at 4° C. overnight. The cells were subsequently post-fixed with 1% osmium tetroxide for 1 h followed by embedding in epoxy resin. The samples were then dehydrated in a series of ethanol washes for 20 min each starting at 70%, 95% and 100%. The samples were then embedded in epoxy resin, placed into moulds, and left to polymerise at 65° C. for 48 h. Sections were taken between 75 and 90 nm on a Leica Ultracut E (Leica, Wetzlar, Germany) and placed on formvar/carbon coated slot grids. Images were observed on a JEOL JEM-1400 Plus TEM, and captured using an AMT UltraVue camera (Woburn, Mass., USA).

Results

Electron microscopy of human exocrine cells reprogrammed according to the protocol containing siARX revealed the presence of dense core granules that were polarised towards one side of the cell (FIG. 17A), a pattern that is typical of islet beta cells.

Higher magnification (FIGS. 17B and 17C) showed the presence of granules, with in some instances a clear dense core surrounded by a non-opaque halo, properties that are characteristic of insulin secretory granules. The dense core of these granules is due to the presence of insulin-zinc hexameric crystalline structures. However, there were also granules that had a less dense core and lacked a halo.

We hypothesised that the lack of zinc in the media could contribute to these intermediate granule forms. This suggested that inclusion of zinc in the media would not only lead to the formation of more dense core secretory granules, but would also enhance the insulin secretory response to glucose and the insulin content of the reprogrammed cells.

Zinc Increases the Level of Insulin mRNA in Reprogramed Cells, Possibly Through a Mechanism that Involves PAX4

To test this hypothesis cells were reprogrammed in the presence or absence of zinc and analysed by RT/QPCR. Cells were reprogrammed (siARX) using the transcription factors and siARX as set out under ‘Methods’. The results demonstrated a significant effect of zinc on insulin gene expression that could in part be attributed to increased levels of mRNA encoding PAX4 (FIG. 18).

Zinc Increases the Insulin (C-Peptide) Content of the Reprogrammed Cells

Further studies showed that Zinc (ZnCl₂) had a stimulatory effect on the insulin (C-peptide) protein content of the reprogrammed (siARX) cells (FIG. 19).

### REFERENCES

Andersson, A., Borg, H., Groth, C. G., Gunnarsson, R., Hellerstrom, C., Lundgren, G., Westman, J., and Ostman, J. (1976). Survival of isolated human islets of Langerhans maintained in tissue culture. J. Clin. Invest. 57, 1295-1301.

Ankrum, J. A., Ong, J. F., and Karp, J. M. (2014). Mesenchymal stem cells: immune evasive, not immune privileged. Nat. Biotechnol.

Banerjee, M., Virtanen, I., Palgi, J., Korsgren, O., and Otonkoski, T. (2012). Proliferation and plasticity of human beta cells on physiologically occurring laminin isoforms. Mol. Cell. Endocrinol. 355, 78-86.

Bar, Y., Russ, H. A., Knoller, S., Ouziel-Yahalom, L., and Efrat, S. (2008). HES-1 is involved in adaptation of adult human beta-cells to proliferation in vitro. Diabetes 57, 2413-2420.

Bar, Y., Russ, H. A., Sintov, E., Anker-Kitai, L., Knoller, S., and Efrat, S. (2012). Redifferentiation of Expanded Human Pancreatic beta-Cell-derived Cells by Inhibition of the NOTCH Pathway. J. Biol. Chem. 287, 17269-17280.

Beattie, G. M., Cirulli, V., Lopez, A. D., and Hayek, A. (1997). Ex vivo expansion of human pancreatic endocrine cells. J. Clin. Endocrinol. Metab. 82, 1852-1856.

Birsoy, K., Chen, Z., and Friedman, J. (2008). Transcriptional regulation of adipogenesis by KLF4. Cell. Metab. 7, 339-347.

Brodsky, J. L., Werner, E. D., Dubas, M. E., Goeckeler, J. L., Kruse, K. B., and McCracken, A. A. (1999). The requirement for molecular chaperones during endoplasmic reticulum-associated protein degradation demonstrates that protein export and import are mechanistically distinct 725. J. Biol. Chem. 274, 3453-3460.

Budd, G. C., Pansky, B., and Glatzer, L. (1993). Preproinsulin mRNA in the rat eye. Invest. Ophthalmol. Vis. Sci. 34, 463-469.

Chan, K. K., Zhang, J., Chia, N. Y., Chan, Y. S., Sim, H. S., Tan, K. S., Oh, S. K., Ng, H. H., and Choo, A. B. (2009). KLF4 and PBX1 directly regulate NANOG expression in human embryonic stem cells. Stem Cells 27, 2114-2125.

Chase, L. G., Ulloa-Montoya, F., Kidder, B. L., and Verfaillie, C. M. (2007). Islet-derived fibroblast-like cells are not derived via epithelial-mesenchymal transition from Pdx-1 or insulin-positive cells. Diabetes 56, 3-7.

Davani, B., Ikonomou, L., Raaka, B. M., Geras-Raaka, E., Morton, R. A., Marcus-Samuels, B., and Gershengorn, M. C. (2007). Human islet-derived precursor cells are mesenchymal stromal cells that differentiate and mature to hormone-expressing cells in vivo. Stem Cells 25, 3215-3222.

Efrat, S., and Russ, H. A. (2012). Making β cells from adult tissues. Trends in Endocrinology & Metabolism 23, 278-285.

Gallo, R., Gambelli, F., Gava, B., Sasdelli, F., Tellone, V., Masini, M., Marchetti, P., Dotta, F., and Sorrentino, V. (2007). Generation and expansion of multipotent mesenchymal progenitor cells from cultured human pancreatic islets. Cell Death Differ. 14, 1860-1871.

Gershengorn, M. C., Hardikar, A. A., Wei, C., Geras-Raaka, E., Marcus-Samuels, B., and Raaka, B. M. (2004). Epithelial-to-mesenchymal transition generates proliferative human islet precursor cells. Science 306, 2261-2264.

Houbracken, I., de Waele, E., Lardon, J., Ling, Z., Heimberg, H., Rooman, I., and Bouwens, L. (2011). Lineage tracing evidence for transdifferentiation of acinar to duct cells and plasticity of human pancreas. Gastroenterology 141, 731-41, 741.e1-4.

Jaager, K., Fatkina, A., Velts, A., Orav, E., and Neuman, T. (2014). Variable expression of lineage regulators in differentiated stromal cells indicates distinct mechanisms of differentiation towards common cell fate. Gene 533, 173-179.

Jonghyeob et al, Expansion and conversion of human pancreatic ductal cells into insulin-secreting endocrine cells, eLIFE, 2013, 2:e00940

Koopmansch, B., Berx, G., Foidart, J., Gilles, C., and Winkler, R. (2013). Interplay between KLF4 and ZEB2/SIP1 in the regulation of E-cadherin expression. Biochem. Biophys. Res. Commun. 431, 652-657.

Lehembre, F., Yilmaz, M., Wicki, A., Schomber, T., Strittmatter, K., Ziegler, D., Kren, A., Went, P., Derksen, P. W., Berns, A., Jonkers, J., and Christofori, G. (2008). NCAM-induced focal adhesion assembly: a functional switch upon loss of E-cadherin. EMBO J. 27, 2603-2615.

Li, R., Liang, J., Ni, S., Zhou, T., Qing, X., Li, H., He, W., Chen, J., Li, F., Zhuang, Q., et al. (2010). A mesenchymal-to-epithelial transition initiates and is required for the nuclear reprogramming of mouse fibroblasts. Cell. Stem Cell. 7, 51-63.

Lima, M. J., Docherty, H. M., Chen, Y., Vallier, L., and Docherty, K. (2012). Pancreatic transcription factors containing protein transduction domains drive mouse embryonic stem cells towards endocrine pancreas. PLoS One 7, e36481.

Lima, M. J., Muir, K. R., Docherty, H. M., Drummond, R., McGowan, N. W., Forbes, S., Heremans, Y., Houbracken, I., Ross, J. A., Forbes, S. J., et al. (2013). Suppression of epithelial-to-mesenchymal transitioning enhances ex vivo reprogramming of human exocrine pancreatic tissue toward functional insulin-producing beta-like cells. Diabetes 62, 2821-2833.

Lin, Z. S., Chu, H. C., Yen, Y. C., Lewis, B. C., and Chen, Y. W. (2012). Kruppel-like factor 4, a tumor suppressor in hepatocellular carcinoma cells reverts epithelial mesenchymal transition by suppressing slug expression. PLoS One 7, e43593.

Liu, Y., Abou-Kheir, W., Yin, J. J., Fang, L., Hynes, P., Casey, O., Hu, D., Wan, Y., Seng, V., Sheppard-Tillman, H., Martin, P., and Kelly, K. (2012). Critical and Reciprocal Regulation of KLF4 and SLUG in Transforming Growth Factor β-Initiated Prostate Cancer Epithelial-Mesenchymal Transition. Molecular and Cellular Biology 32, 941-953.

McCall, M., and Shapiro, A. M. (2012). Update on islet transplantation. Cold Spring Harb Perspect. Med. 2, a007823.

Morton, R. A., Geras-Raaka, E., Wilson, L. M., Raaka, B. M., and Gershengorn, M. C. (2007). Endocrine precursor cells from mouse islets are not generated by epithelial-to-mesenchymal transition of mature beta cells. Mol. Cell. Endocrinol. 270, 87-93.

Muir, K. R., Lima, M. J., Docherty, H. M., and Docherty, K. (2014). Cell therapy for type 1 diabetes. QJM 107, 253-259.

Nielsen, J. H., Brunstedt, J., Andersson, A., and Frimodt-Moller, C. (1979). Preservation of beta cell function in adult human pancreatic islets for several months in vitro. Diabetologia 16, 97-100.

Ohgushi, M., Matsumura, M., Eiraku, M., Murakami, K., Aramaki, T., Nishiyama, A., Muguruma, K., Nakano, T., Suga, H., Ueno, M., et al. (2010). Molecular pathway and cell state responsible for dissociation-induced apoptosis in human pluripotent stem cells. Cell. Stem Cell. 7, 225-239.

Ouziel-Yahalom, L., Zalzman, M., Anker-Kitai, L., Knoller, S., Bar, Y., Glandt, M., Herold, K., and Efrat, S. (2006). Expansion and redifferentiation of adult human pancreatic islet cells. Biochem. Biophys. Res. Commun. 341, 291-298.

Rooman, I., Heremans, Y., Heimberg, H., and Bouwens, L. (2000). Modulation of rat pancreatic acinoductal transdifferentiation and expression of PDX-1 in vitro. Diabetologia 43, 907-914.

Russ, H. A., Bar, Y., Ravassard, P., and Efrat, S. (2008). In vitro proliferation of cells derived from adult human beta-cells revealed by cell-lineage tracing. Diabetes 57, 1575-1583.

Russ, H. A., Ravassard, P., Kerr-Conte, J., Pattou, F., and Efrat, S. (2009). Epithelial-mesenchymal transition in cells expanded in vitro from lineage-traced adult human pancreatic beta cells. PLoS One 4, e6417.

Ryan, E. A., Paty, B. W., Senior, P. A., Bigam, D., Alfadhli, E., Kneteman, N. M., Lakey, J. R., and Shapiro, A. M. (2005). Five-year follow-up after clinical islet transplantation. Diabetes 54, 2060-2069.

Samavarchi-Tehrani, P., Golipour, A., David, L., Sung, H. K., Beyer, T. A., Datti, A., Woltjen, K., Nagy, A., and Wrana, J. L. (2010). Functional genomics reveals a BMP-driven mesenchymal-to-epithelial transition in the initiation of somatic cell reprogramming. Cell. Stem Cell. 7, 64-77.

Shapiro, A. M., Lakey, J. R., Ryan, E. A., Korbutt, G. S., Toth, E., Warnock, G. L., Kneteman, N. M., and Rajotte, R. V. (2000). Islet transplantation in seven patients with type 1 diabetes mellitus using a glucocorticoid-free immunosuppressive regimen 4. N. Engl. J. Med. 343, 230-238.

Skyler, J. S. (2013). Primary and secondary prevention of Type 1 diabetes. Diabet. Med. 30, 161-169.

Takahashi, K., and Yamanaka, S. (2006). Induction of pluripotent stem cells from mouse embryonic and adult fibroblast cultures by defined factors. Cell 126, 663-676.

Tiwari, N., Meyer-Schaller, N., Arnold, P., Antoniadis, H., Pachkov, M., van Nimwegen, E., and Christofori, G. (2013). Klf4 is a transcriptional regulator of genes critical for EMT, including Jnk1 (Mapk8). PLoS One 8, e57329.

Weinberg, N., Ouziel-Yahalom, L., Knoller, S., Efrat, S., and Dor, Y. (2007). Lineage tracing evidence for in vitro dedifferentiation but rare proliferation of mouse pancreatic beta-cells. Diabetes 56, 1299-1304.

White, M. G., Al-Turaifi, H. R., Holliman, G. N., Aldibbiat, A., Mahmoud, A., and Shaw, J. A. M. (2011). Pluripotency-associated stem cell marker expression in proliferative cell cultures derived from adult human pancreas. Journal of Endocrinology 211, 169-176.

Yori, J. L., Johnson, E., Zhou, G., Jain, M. K., and Keri, R. A. (2010). Kruppel-like factor 4 inhibits epithelial-to-mesenchymal transition through regulation of E-cadherin gene expression. J. Biol. Chem. 285, 16854-16863.

Zanini, C., Bruno, S., Mandili, G., Baci, D., Cerutti, F., Cenacchi, G., Izzi, L., Camussi, G., and Forni, M. (2011). Differentiation of mesenchymal stem cells derived from pancreatic islets and bone marrow into islet-like cell phenotype. PLoS One 6, e28175.

Zhang, P., Andrianakos, R., Yang, Y., Liu, C., and Lu, W. (2010). Kruppel-like factor 4 (Klf4) prevents embryonic stem (ES) cell differentiation by regulating Nanog gene expression. J. Biol. Chem. 285, 9180-9189

Zhang et al, Functional Role of an Islet Transcription Factor, INSM1/IA-1, on Pancreatic Acinar Cell Trans-Differentiation, J. Cell. Physiol. 227: 2470-2479, 2012

1. A method of producing an expanded population of pancreatic cells, the method comprising:
(i) providing a starting pancreatic cell population;
(ii) culturing the starting pancreatic cell population under a first condition that promotes expansion and dedifferention of the starting pancreatic cell population;
(iii) culturing the dedifferentiated cells obtained in step (ii) under a second condition which induces redifferentiation;
(iv) thereby obtaining an expanded population of redifferentiated pancreatic cells, wherein said second condition comprises treating the cells with exogenous KLF4.

2. The method according to claim 1, wherein the starting pancreatic cell population is a human pancreatic material comprising a mixed population of cells, the mixed population of cells including beta-cells and at least one other type of pancreatic epithelial cells.

3. The method according to claim 1, wherein the starting pancreatic cell population is an islet enriched fraction from human pancreas.

4. The method according to claim 1, wherein the expanded population of redifferentiated pancreatic cells comprises pancreatic cells which are insulin expressing cells.

5. The method according to claim 1, wherein the first condition comprises the use of adherent culture.

6. The method according to claim 1, wherein the second condition comprises the use of suspension culture.

7. The method according to claim 1, wherein the second condition comprises the use of serum-containing media.

8. The method according to claim 1, wherein the culturing in step (iii) is carried out for 4 or more days.

9. The method according to claim 1, wherein the exogenous KLF4 is KLF4 protein or a nucleic acid which expresses KLF4 in the cells.

10. The method according to claim 9, wherein the nucleic acid is introduced into the cells using an adenovirus vector.

11. The method according to claim 1, wherein the second condition further comprises treating the cells with one or more exogenous factors selected from the list consisting of: FOXA1, FOXA2, PDX1, NGN3, PAX4, MAFA, NKX6.1, NKX2.2, NEUROD1, PAX6, IA-1 and GATA4 or a nucleic acid which expresses one or more of the foregoing exogenous factors.

12. The method according to claim 11, wherein the second condition comprises treating the cells with one FOXA1 or FOXA2 following treatment with KLF4 or a nucleic acid which expresses one or more of the foregoing exogenous factors.

13. The method according to claim 1, wherein the second condition further comprises treatment with zinc.

14. An expanded population of redifferentiated pancreatic cells obtained or by the method according to claim 1.

15. The population as claimed in claim 14, wherein the expanded cells express INS, SST or both.

16. The population as claimed in claim 14, where at least 50% of the cells in the expanded cell population obtained express an epithelial marker.

17. A method of inducing redifferentiation of pancreas-derived mesenchymal stromal cells, comprising contacting a population of pancreas-derived mesenchymal stromal cells with KLF4, or a nucleic acid encoding KLF4.

18. (canceled)

19. A method of treatment of diabetes in a patient, comprising administering theef expanded population of redifferentiated pancreatic cells according to claim 14, wherein at least a portion of the population of cells is capable of expressing insulin.

20. The method of treatment according to claim 19, wherein administering comprises transplanting the pancreatic cells into the patient.

21. The method of treatment according to claim 20, wherein transplanting is done with one or more immunosuppressants.

22. The method of treatment according to any one of claim 19, wherein the diabetes is type-1-diabetes.

23. (canceled)

24. (canceled)

25. (canceled)

26. A kit for performing a method of claim 1, said kit comprising:
(i) KLF4 or a nucleic acid encoding KLF4; and one or more of:
(ii) a transcription factors selected from FOXA1, FOXA2, PDX1, NGN3, PAX4, MAFA, NKX6.1, NKX2.2, NEUROD1, PAX6, IA-1 and GATA4, or a nucleic acid encoding one or more of the foregoing transcription factors;
(iii) written instructions for use in said method.
